(19) United States
(12) Patent Application Publication
(10) Pub. No.: US 2024/0115781 A1
(43) Pub. Date: Apr. 11, 2024

(54) DESIGN AND OPERATIONAL FEATURES FOR HIGH EFFICIENCY HIGH HEMOCOMPATIBILITY MICROFLUIDIC RESPIRATORY SUPPORT DEVICE

(71) Applicant: The Charles Stark Draper Laboratory, Inc., Cambridge, MA (US)

(72) Inventors: Brett Isenberg, Newton, MA (US); Else Vedula, Stoneham, MA (US); David Sutherland, Belmont, MA (US); Diana Lewis, Cambridge, MA (US); Jose Santos, Westwood, MA (US); WeiXuan Lai, Weymouth, MA (US); Ernie Kim, Cambridge, MA (US); Beau Landis, Boston, MA (US); Jeffrey Borenstein, Boston, MA (US); Bryan Teece, Mattapoisett, MA (US); Samuel Blumenstiel, Boston, MA (US); Joseph Urban, Cambridge, MA (US)

(73) Assignee: The Charles Stark Draper Laboratory, Inc., Cambridge, MA (US)

(21) Appl. No.: 18/376,318

(22) Filed: Oct. 3, 2023

Related U.S. Application Data
(60) Provisional application No. 63/413,227, filed on Oct. 4, 2022.

(51) Int. Cl. A61M 1/16

(52) U.S. Cl. CPC A61M 1/1621

(57) ABSTRACT

Systems and apparatuses for blood oxygenation are disclosed. A system includes a first layer defining a plurality of banks of first channels each extending in a first direction. The plurality of banks of first channels are configured to receive blood via a trunk channel. The system includes a second layer defining a bank of second channels extending in a second direction. The bank of second channels are configured to receive oxygen. The first direction is different from the second direction. The system includes a membrane disposed between the first layer and the second layer and configured to cause the oxygen to permeate from the second layer to the first layer to oxygenate the blood.

### CROSS-REFERENCES TO RELATED APPLICATIONS

This application claims the benefit of and priority to U.S. Provisional Patent Application No. 63/413,227, filed Oct. 4, 2022, the contents of which is incorporated herein by reference in its entirety for all purposes.

### BACKGROUND

Extracorporeal membrane oxygenation (ECMO) is a procedure used to oxygenate blood. ECMO includes establishing a large bore vascular access for either venovenous or arteriovenous circulation of the blood. Gas exchange may occur in a hollow fiber membrane oxygenator (HFMO) specially designed for high efficiency gas transfer. However, in spite of high efficiency gas transfer and improvements in membrane materials and hemocompatible surface coatings and cartridge housing designs, ECMO remains a dangerous procedure with many complications associated with the blood circuit, principally related to clotting and bleeding.

### SUMMARY

Microfluidic oxygenators may be utilized to address some of the issues relating to blood clotting and bleeding in ECMO procedures. Microfluidic oxygenation has a number of advantages, including the ability to fashion smaller channels with thinner gas transfer membranes than HFMO, which can lead to higher efficiency gas transfer. Microchannel networks also have the ability to recapitulate key aspects of the architecture of vascular circulation in the body, far more so than a hollow fiber device. The devices described herein demonstrate improved hemocompatibility due to reduced blood disturbances in the microchannel networks, and provide a scalable parallel design that can enable use at clinically relevant blood flow rates. The systems described herein provide improved hemocompatibility while also enabling significant scaling to clinically relevant blood flow rates, without the inherent drawbacks of HFMO-based approaches.

At least one aspect of the present disclosure is directed to an ECMO device. The ECMO device includes a first layer defining a plurality of banks of first channels each extending in a first direction. The plurality of banks of first channels can be configured to receive blood via a trunk channel. The ECMO device includes a second layer defining a bank of second channels extending in a second direction. The bank of second channels can be configured to receive oxygen. The ECMO device includes a membrane disposed between the first layer and the second layer and configured to cause the oxygen to permeate from the second layer to the first layer to oxygenate the blood.

In some implementations, at least one bank of the plurality of banks of first channels is configured to receive the blood via a finger channel in fluid communication with the trunk channel. In some implementations, the finger channel has a taper along a length of the at least one bank. In some implementations, the at least one bank comprises a plurality of bifurcating channels in fluid communication with the finger channel. In some implementations, each of the plurality of bifurcating channels bifurcate at least twice.

In some implementations, the trunk channel comprises at least one trunk ramp configured to maintain shear stress on the blood within a predetermined range as the blood flows through the plurality of banks of channels. In some implementations, the bank of second channels is configured to receive the oxygen via an oxygen channel having a taper along a length of the bank of second channels. In some implementations, the oxygen channel comprises one or more support structures. In some implementations, the membrane has a thickness ranging from about 50 μm to about 100 μm. In some implementations, the membrane comprises polydimethylsiloxane (PDMS). In some implementations, the plurality of banks of first channels comprise four banks of first channels, each receiving oxygen from the bank of second channels via the membrane.

At least one other aspect of the present disclosure is directed to a system. The system includes a housing comprising a plurality of oxygenator layers. Each oxygenator layer includes a bank of first channels configured to receive blood, a bank of second channels configured to receive oxygen, and a membrane configured to cause the oxygen to permeate from the bank of second channels to the banks of first channels to oxygenate the blood. The system includes a vertical manifold configured to provide the blood to each of the plurality of oxygenator layers.

In some implementations, the vertical manifold is configured to maintain a range of shear stress in the blood ranging from about 7 dynes/cm2 to about 35 dynes/cm2. In some implementations, the vertical manifold is further configured to provide the oxygen to the plurality of oxygenator layers. In some implementations, the housing comprises a plurality of trays each having a respective oxygenator layer of the plurality of oxygenator layers. In some implementations, the vertical manifold is coupled to the plurality of trays. In some implementations, the system includes an oxygen source configured to provide the oxygen at a predetermined pressure. In some implementations, the vertical manifold comprises a plurality of tubes positioned within a casing.

Yet another aspect of the present disclosure is directed to a layer of an ECMO device. The layer includes an inlet configured to receive blood. The layer includes a trunk channel in fluid communication with the inlet. The layer includes a plurality of finger channels extending from the first channel. The layer includes a plurality of banks of blood channels. Each of the plurality of banks of blood channels is in fluid communication with a respective one of the plurality of finger channels via a bifurcating manifold configured to maintain shear stress on the blood within a predetermined threshold.

In some implementations, a shape of one or more curves of the plurality of finger channels is configured to maintain shear stress on the blood within a predetermined threshold. In some implementations, each of the plurality of banks of blood channels are in fluid communication with an outlet channel via a second bifurcating manifold. In some implementations, the outlet channel is parallel to the trunk channel.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form.

### BRIEF DESCRIPTION OF THE DRAWINGS

FIG. 1A is a top view of an example blood layer of an ECMO system with four parallel banks of oxygenation channels, according to an embodiment;

FIG. 1B is a zoomed perspective view of the bifurcating channels of two parallel banks of oxygenation channels of the blood layer shown in FIG. 1A, according to an embodiment;

FIG. 1C is a perspective view of an example oxygen layer that may be utilized with the oxygenation layer shown in FIGS. 1A-1D, according to an embodiment;

FIG. 1D is a zoomed perspective view of the oxygen-carrying channels of the example oxygen layer shown in FIG. 1C, according to an embodiment;

FIG. 2 is a top view of a portion of an example blood layer similar to the ECMO system blood layer shown in FIG. 1A that is oxygenating blood, according to an embodiment;

FIG. 3 is a photograph comparing a single bank design of a blood-containing layer to a multi-bank design of a blood layer with multiple inlets and outlets, according to an embodiment;

FIG. 4 is a diagram of a perspective view of an example trunk ramp of the device shown in FIG. 1A, according to an embodiment;

FIGS. 5A and 5B are photographs showing heatmaps of the shear stress experienced by fluid when flowing through an alternative trunk ramp design and the trunk ramp of FIG. 4, respectively, according to an embodiment;

FIG. 6 is a close-up photograph of the bifurcating channels of the device shown in FIGS. 1A and 1B, according to an embodiment;

FIGS. 7A and 7B show three-dimensional (3D) metrology images of blood layer channels with color overlays corresponding to measured dimensions, according to an embodiment;

FIGS. 8A, 8B, and 8C show example views of a device similar to that shown in FIG. 1A with example membrane support features in the oxygen-carrying layer, according to an embodiment;

FIG. 9 shows example block diagrams showing the application of different pressures on a membrane in a device similar to that shown in FIG. 1A, according to an embodiment;

FIG. 10 shows a diagram of an example system in which a pressure controller can apply oxygen backpressure in a device similar to that shown in FIG. 1A to improve oxygenation of blood flowing through the device, according to an embodiment;

FIGS. 11A and 11B show perspective and zoomed views, respectively, of an example device similar to that shown in FIG. 1A that is oxygenating blood, according to an embodiment;

FIGS. 12A and 12B show an example tray and housing, respectively, which may be utilized to house one or more layers similar to the layers shown in FIG. 1A, according to an embodiment;

FIGS. 13A and 13B show cross-sectional photographs of a single-membrane design for a device similar to that shown in FIG. 1A, according to an embodiment;

FIGS. 14A, 14B, and 14C show photographs of an example vertical manifold that may be utilized with multiple parallel layers of the device shown in FIG. 1A, according to an embodiment;

FIG. 15 is a graph showing thrombus accumulation relative to a control for coated and uncoated devices, according to an embodiment;

FIG. 16A shows a diagram illustrating a process for manufacturing an example dual-membrane device that is similar to the single-membrane devices described herein, according to an embodiment;

FIG. 16B shows a diagram illustrating another example process for manufacturing an example dual-membrane device that is similar to the single-membrane devices described herein, according to an embodiment;

FIGS. 17A and 17B show photographs of an example dual-membrane device with two oxygen-carrying layers, according to an embodiment;

FIGS. 18A and 18B show cross-sectional photographs of a dual-membrane as described herein, according to an embodiment;

FIGS. 19A, 19B, 19C, and 19D show example photographs of an example manufacturing process for the dual-membrane devices described herein, according to an embodiment;

FIGS. 20A, 20B, and 20C show perspective diagrams of an example edge manifold with rectangular ducts, according to an embodiment;

FIGS. 21A, 21B, and 21C show perspective diagrams of interior space constructs used in a manufacturing process for an alternative edge manifold, according to an embodiment;

FIGS. 22A and 22B show example views of an example process for manufacturing the alternative edge manifold using the interior space constructs shown in FIGS. 21A, 21B, and 21C, according to an embodiment;

FIGS. 23A and 23B show additional example views of the example process for manufacturing the alternative edge manifold using the interior space constructs shown in FIGS. 21A, 21B, and 21C, according to an embodiment;

FIGS. 24A, 24B, and 24C show the final steps in the example process for manufacturing the alternative edge manifold, according to an embodiment;

FIGS. 25A and 25B show views of example ducts that may be utilized as an edge manifold for a device similar to that shown in FIG. 1A, according to an embodiment;

FIGS. 26A and 26B show graphs of example experimental results from tests measuring oxygen transfer at different blood flow rates and oxygen back pressures, according to an embodiment;

FIGS. 27A and 27B show graphs of example experimental results from tests measuring blood pressure at different blood flow rates and oxygen back pressures, according to an embodiment;

FIGS. 28A and 28B show graphs of example experimental results from tests measuring flow rate and oxygen transfer, respectively, over time in animal studies, according to an embodiment; and

FIGS. 29A, 29B, and 29C show graphs of example experimental results indicating blood health from animal tests, according to an embodiment.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The techniques described herein provide systems and apparatuses for ECMO that utilize a parallel multi-bank microfluidic design. The use of microfluidics in ECMO can be used to overcome many of the limitations of traditional HFMO cartridges in terms of blood health by recapitulating key aspects of the of the microcirculation such as vessel dimensions designed for optimum oxygen transfer and minimal pressure drop as well as flow patterns that allow for smooth transitions between large high-flow distribution channels and the small, low-flow channels where oxygen transfer primarily occurs. Approaches to ECMO that do not utilize the techniques described herein lack controlled, uniform shear stress at all length scales, scalable operation at clinically relevant flow rates, and stabile, long-term performance. The systems and apparatuses described herein address these challenges and other challenges by utilizing a multi-banked layer design with particular geometry to reduce blood shear stress.

The multi-layer systems and apparatuses described herein can provide at least 750 mL/min of throughput without significant pressure drop across each layer. With this increased throughput, our system can operate range required for clinical relevance for patients. The throughput of the multi-layer approach offers can be increased by simply adding additional parallel layers to the device. The systems and apparatuses described herein can be fabricated using high-volume manufacturing processes, such as silicone injection molding, which allow for the generation of layers on the order of 3-4 mm thick and can reduce overall device size. Layers in which channels are molded onto both sides can be further reduced by removing redundant structural materials between layers. Embodiments including channel geometry that allows for oxygen transfer membranes to be placed on both sides of the blood channel can reduce the overall device size by approximately 50%. With these improvements, a microfluidic EMCO device based upon the present techniques and operating at a clinically relevant flow rate of 5 L/min can be manufactured under 8 L in size.

One feature of ECMO devices is the ability to operate consistently over extended periods of time without failure of the device or doing harm to the patient, both of which include creating a flow path that amenable to high oxygen transfer efficiency and is designed with blood health in mind that is tolerant to perturbations, fouling and clogging. The techniques presented herein maintain uniform shear stress within a physiological range throughout the flow path, which improves blood health outcomes in ECMO devices even in the absence of anti-thrombogenic coating or heavy use of anticoagulants. The inlet and outlet regions of devices not implementing the present techniques that interface with external tubing via stainless steel hypotubes were areas where the blood was exposed high shear stress gradients, which are known to adversely affect blood health, as it transitions from a circular tube to a rectangular duct. The present techniques provide systems and apparatuses that include particular sizing and shaping for each area of the flow path that ensures the blood is exposed to a very narrow range of shear stress (7-35 dynes/cm\hat{ }2) at nearly all points in the flow path with no sharp gradients or oscillations.

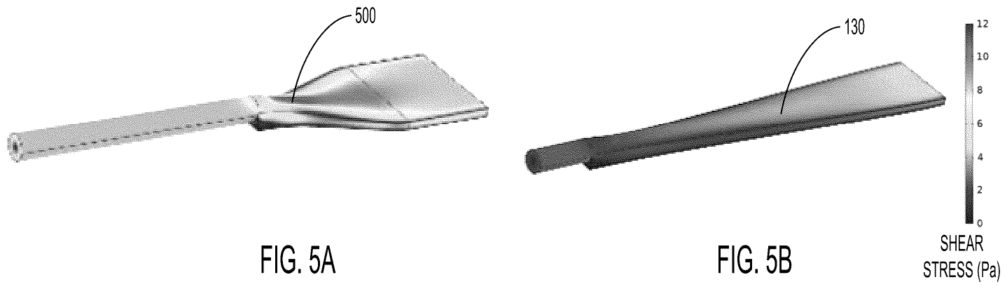

Although platelet activation and sensitization were considered as a driving factor in determining the ramp geometry and the total shear load of the device, the peak shear stress limit was set by the desire to remain in a thrombin dominant platelet aggregation regime (<35 dynes/cm2) rather than a vWF regime (>50-100 dynes/cm2). The thrombin regime allows the systems described herein to limit platelet aggregation with heparin-based strategies, both systemic dosage and bound surface coatings. The lower limit of 7dynes/cm2 allows the systems described herein to reduce material induced trauma or recirculation-like effects which exhibit as increased compliment system activation. The cross-section of each portion of the flow path was chosen to meet the above criteria and the shape of transition regions between channels of different dimensions was achieved via an iterative process involving analytical estimates of shear stress, updating of the 3D computer-aided design (CAD) model and verification with computational fluid dynamics (CFD) in order to carefully sculpt these regions to avoid any sudden changes in shear. These approaches resulted in improvements to the layer inlet and outlet transition areas, as shown in the comparison of FIGS. 5A and 5B. The systems and apparatuses described herein implement a large number of shorter channels arranged in multiple banks that, despite the greatly increased flow rate, keeps the pressure drop to a clinically appropriate level while making each layer more tolerant to channel blockages by providing a large number of potential pathways for the blood to redistribute through without significantly altering the flow through any individual channel or region of the layer.

Soft lithography, which can serve as the basis for some microfluidic ECMO devices, provides tremendous utility and versatility for creating microfluidic devices; however, it is primarily limited to generating structures that are two-dimensional (2D) in nature. To create the smooth, gradual transitions between channels of different length scales as described herein, three-dimensional structures are utilizing. In an embodiment, 3D-printing may provide the resolution and dimensional stability large enough areas multiple length scales to manufacture the systems described herein. One other manufacturing technique includes high-precision micromachining, which offers the ability to generate molds with the complex 3D channel geometries as described herein. Such techniques are utilized to fabricate high-resolution master molds (e.g., of a suitable material such as gold-coated aluminum) that are used to cast individual blood and oxygen layers.

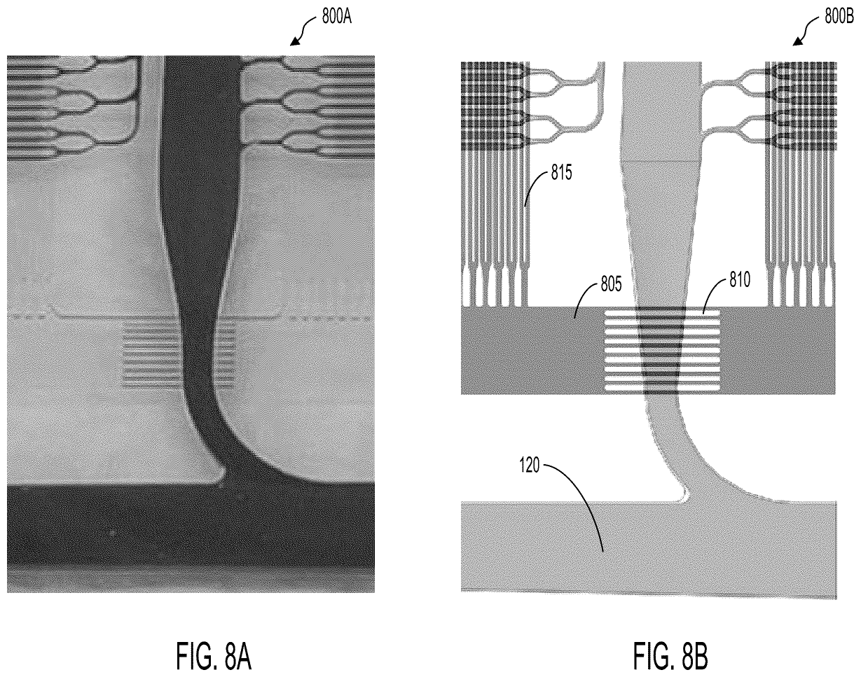

Using the techniques described herein, increasing the pressure of oxygen sweep gas through the device can be an effective approach to enhancing oxygenation performance. The systems described herein do not result in adverse effects of increased pressure drop during short-term and long-term in vitro oxygenation tests. To address the issue of pressure drops, the oxygen-carrying layers of the mold were designed to reduce the dimensions of the overlap area in each channel without decreasing the overall transfer area by including a large number of oxygen channels. This minimizes the membrane deflection to an acceptable level at less than 100 mmHg at 100 mL/min blood flow when oxygen backpressure is applied, resulting in a greatly reduced pressure drop across the device at the nominal flow rate of 100 mL/min. These techniques reduce deflection to a level that will present little risk to future experiments or patients. Additionally, support structure as shown in FIGS. 8A, 8B, and 8C can be used to minimize membrane deflection under pressure. These approaches allow for greater oxygenation efficiency at low-pressure drop. Example experimental results of an example implementation of the present techniques are provided below in Table 1.

TABLE 1

| Layer | | | | | | | |
|---|---|---|---|---|---|---|---|
| flow rate at 5 | | | | | Transfer | | |
| vol % | | | | | area | Prime | |
| [mL/ | | | Pressure | | exchange | Volume per | in vivo Study |
| min/ | | | drop | Layers for | efficiency ^{a)} | Layer per | Duration |
| layer] | Sweep gas | Vol % ^{a)} | [mmHg] | 1 L/min | [mL/min/m²] | mL/min [s] | [hr] |
| 90 | 100% oxygen | 6.7% | 122 | 11 | 308 (264)** | 9 | 24 |
| | at 400 | (5.0%) | | | | | |
| | mmHg, | | | | | | |
| | 100 | | | | | | |
| | mL/min | | | | | | |

To aid in the design of the devices and systems described herein, models (e.g., COMSOL models) were established using data from devices with varying thickness membranes and backpressures. The model was tuned to match a desired performance by adjusting the membrane permeability and using an apparent oxygen diffusivity and solubility in blood. Because devices were tested that had different channel heights (65-200 μm), lengths (4.7-8 cm), and various measured membrane thicknesses (50-100 μm), and were tested at different backpressures, the oxygen diffusivity and solubility of both the membrane and the blood could be independently back-calculated to create a model that precisely matched the data from all testing. The channel widths constrained to 500 μm.

This model was then run with a series of channel lengths, heights, number of channels, membrane thicknesses, backpressures, and flow rates. The outputted oxygen transfer from each condition was then plotted to determine trends. The results indicated that a total channel length (number of channels times the channel length) determined the oxygen transfer, significantly simplifying device design. The total channel length was plotted and an equation extracted for vol % oxygen transfer vs. total length. For a given membrane thickness and desired vol % transfer, a polynomial curve fit was used between data points to determine the required total channel length for a given channel height and flow rate. The target oxygen transfer and flow rate per layer were set at 5 vol % at 100 mL/min, respectively. Because higher backpressure results in a smaller overall layer size due to the higher rate of oxygen transfer, the device was based on the use of 400 mmHg backpressure.

To determine the maximum length of an individual channel (and therefore determine the number of channels required as Lₜₒₜₐₗ/L_{channel}) for a given flow rate and channel height, a maximum pressure drop of 50 mmHg per channel (excluding manifolds) was set. The maximum length of a channel was then calculated using the formula:

$$Δ P = \frac{12 η L Q}{( 1 - 0.63 \frac{h}{w} ) {h}^{3} w} ,$$

with number of channels calculated as described herein.

As many channel heights and lengths fit this criteria, a further design constraint was added; to minimize the device area. Using example values for a maximum device width of 10 inches and assuming 400 μm between channels, a number of channels per width was calculated, and a number of banks calculated. This resulted in designs to that can include three, four, or five banks, all which had very similar minimum areas. Four banks were chosen as the final design due to the possibility of keeping a bifurcating manifold. However, it should be understood that alternative design constraints can be utilized to arrive at different designs that similarly utilize the techniques described herein to oxygenate blood using microfluidic channels. An example device length can be calculated as follows:

L_{device}=2*(1.35 cm outer)+(N_{bank}−1)*2.25 cm+N_{bank}*L

This example design was modeled at various flow rates and backpressures to predict device performance at other backpressures and flow rates.

To maintain the hemocompatibility the design, an additional set of rules were applied across the device to optimize the flow-induced shear stresses imparted on the blood cells and key plasma proteins. As shear forces increase, so does the risk of activating platelets, enabling shear-modulated platelet binding regimes, and triggering the release of pro-thrombotic agents. Alternatively, low shear rates, caused by recirculation or low flow rates, can result in stasis-like conditions or prolonged exposure to the device's foreign material surfaces, which promote platelet aggregation and the release of pro-inflammatory factors. Balancing these effects can minimize the thrombogenicity of the channel design and eliminates the presence of cell damaging shear forces which occur far above the shear thresholds discussed here. Conservative thresholds were chosen for the design's total shear stress load and transitional shear ramp rate; 1000 dyne*s/cm2 for durations greater than 10 seconds and <170 dynes/cm2/s respectively. A maximal shear stress target of 35 dynes/cm2 was set to minimize von Wilbrand factor modulated platelet aggregation and maintain a thrombin dominate regime that can be addressed with a heparin-based anticoagulation strategy. The targeted lower shear stress limit was 7 dynes/cm2 was applied to limit compliment activation.

The systems described herein can utilize external manifolds to connect individual layers in a multi-layer embodiment. For example, an 8-layer device, as described herein, can utilize a three-level bifurcating manifold (1-to-8). Murray's law can be used to define the branch diameters using the inner diameter of layer entrance and exit ports as the basis for sizing the other channels and the same shear stress target range described above for the individual oxygenation layers. An iterative CAD-CFD process can be used to shape the transition from one level to the next in order to minimize low-flow regions and shear gradients.

The manufacturing techniques for the approaches described herein can utilize molds for casting the vascular and oxygen channel networks. The molds can be created by milling the negative of the designed channel architecture described herein into a block of a suitable material (e.g., aluminum, another metal material, a composite material, etc.). A CNC machine using various end mills can be used to remove material from the block and leave behind the trunk manifold and channel structure. The blocks can then be gold sputtered coated. The molds for the manifolds can also milled out of a suitable using similar techniques. Tapped holes can placed in the mold at a number of different areas to allow for pins to be placed in the mold in order to allow for alignment features to be cast into the manifold casted parts. The manifold molds can be similarly gold sputter coated. Additional manufacturing techniques can also be utilized to fabricate the layers described herein, including, but not limited to, silicone injection molding, roll-to-roll processing, or roller-based methods, among others.

Each layer in the multi-layer design can include a vascular channel part and an oxygen channel part with a PDMS membrane sandwiched between the two. The membrane can be fabricated using any suitable material, including Silpuran (Wacker Chemie AG of Munich, Germany). The vascular and oxygen parts were cast using NuSil MED6015 (Avatar, Santa Barbara, CA) into each mold. The MED6015 can be prepared by mixing the PDMS with curing agent in a 10:1 ratio per manufacturer specifications. The molds containing the PDMS can be placed into a vacuum chamber in order to remove any trapped air bubbles. The parts were placed in a 65 C oven to cure. Manifold parts were cast using the same method described above. MED6015 can be used in each manifold mold in order to create the two halves of the distribution manifold.

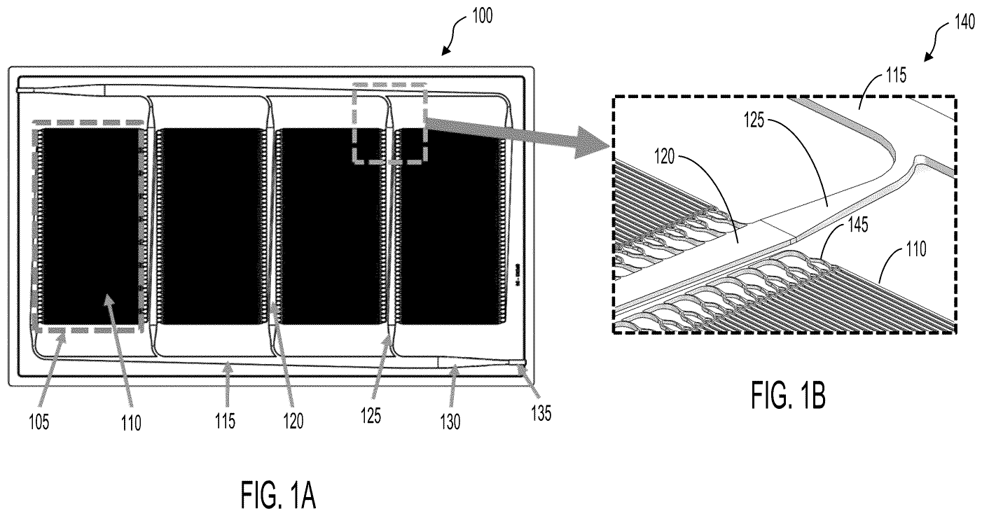

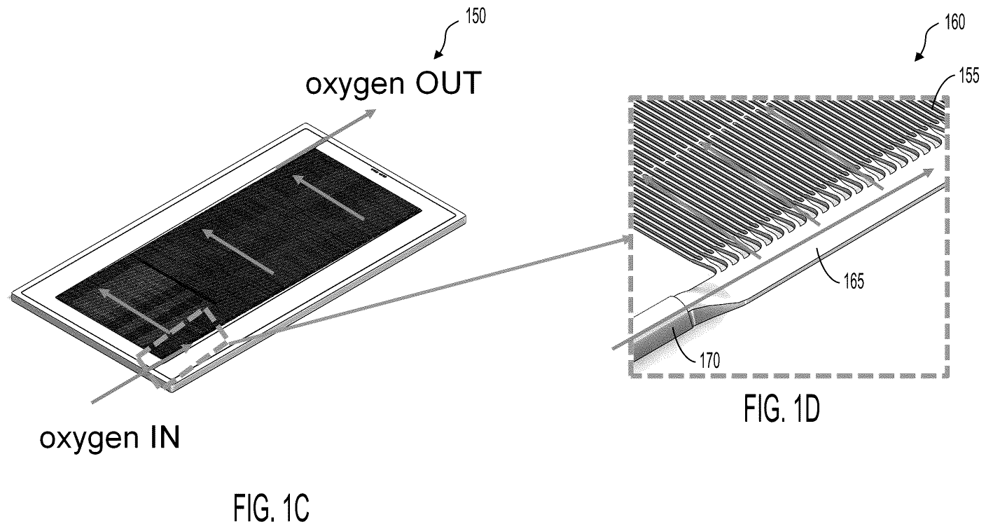

Referring to FIGS. 1A and 1B, illustrated is a top view of a blood layer of an example ECMO system 100 with four parallel banks 105 of oxygenation channels 110; and a zoomed perspective view 140 of bifurcating channels 145 of two parallel banks of oxygenation channels 110, according to an embodiment. The system 100 can include the blood layer coupled to a membrane and an oxygen layer (described in connection with FIGS. 1C and 1D). The membrane can separate the blood layer from the oxygen layer. In some implementations, the ECMO system 100 can include one or more blood layers, oxygen layers, and membranes. A combined oxygenation layer of the system 100 can include two molded halves: one for blood and one for oxygen, separated by a gas permeable membrane. Multiple combined oxygenation layers may be utilized in parallel to increase throughput, as described herein. Particular configurations for the oxygenation channels 110 were used for a single-inlet 135 and single-outlet blood layer capable of achieving a target performance specification of 5 vol % oxygen transfer at a flow rate of 100 mL/min/layer. Additional design constraints included a maximum 50 mmHg pressure drop, 3.5 Pa maximum shear stress, 50 μm thick membrane, and 10 in maximum layer width. This effectively limited the number of parallel channels that span the layer to 184, allowing room for distribution channels and entrance/exit regions. Increasing the total number of channels improves overall redundancy such that individual clogged channels will have less impact on overall performance.

As shown, the oxygenation layer (sometimes referred to as the “blood layer” or the “blood containing layer”) includes a four banks 105 of channels 110. Four banks are utilized because that number is compatible with a bifurcating manifold network. The example layer includes 736 individual oxygenation channels 110. Each oxygenation channel 110 can have varying dimension, for example, 9.1 cm long, 500 μm wide, and 160 μm deep. However, other material ranges are possible (e.g., 7 cm to 10 cm long, 250 μm to 750 μm wide, 50 μm to 200 μm deep, etc.). Within each bank, the oxygenation channels 110 are connected upstream and downstream by a two-level bifurcating channel network (e.g., the bifurcating channels 145 of FIG. 1B) to large distribution channels referred to as “fingers” 120. The fingers 120 then connect with an even larger distribution channel referred to as a “trunk” 115. As shown in FIG. 1B, the fingers 120 can connect with the trunk via a smooth transition (shown here as a curve), such that the shear stress on the blood is maintained within a predetermined range (e.g., between about 7 dynes/cm² to about 35 dynes/cm²). The trunk lines interface directly with the entrance port 135 and exit port of the layer via corresponding trunk ramps 130. At each convergence point or change in channel dimensions, carefully engineered transitions regions (e.g., the finger ramp 125, the bifurcating channels 145 of FIG. 1B, etc.) allow the flow to be maintained in a narrow range of shear stress as described herein. In this example, the volume of the oxygenation channels 110 is 14.7 mL, with a residence time of 8.8 s at 100 mL/min. The example layer dimensions are 253 mm wide by 478 mm long. However, it should be understood that other layer dimensions, volume, and residence time are also possible.

A multi-banked design of the system 100 allows for a greater number of short channels within a transfer area, with the main benefit of reducing overall pressure drop during blood flow. This is opposed to a single bank of long channels that span the footprint of the device layer. The multi-banked design of the system 100 was a result of an iterative design procedure that effectively limited the number of parallel channels that span the vascular layer (e.g., the blood layer) to 184, allowing room for distribution channels and entrance/exit regions. Increasing the total number of channels improves overall redundancy such that individual clogged channels will have less impact on overall performance.

Referring to FIGS. 1C and 1D, illustrated is a perspective view of an example oxygen layer 150 that may be utilized with the blood layer of the system 100 shown in FIG. 1A; and a zoomed perspective view 160 of the oxygen-carrying channels 155 of the example oxygen layer 150 shown in FIG. 1C, according to an embodiment. The oxygen layer 150 can ensure uniform oxygen distribution across the network to provide optimum oxygen transfer at all points. Unlike the blood layer of FIGS. 1A and 1B, the oxygen layer 150 includes a single large bank of parallel channels 155 that run perpendicular to the blood oxygenation channels 110 and connect to a corresponding entrance port 170 and a corresponding exit port via a trunk line 165, similar to the blood side. This arrangement allows for a balance between oxygen transfer area and support for the membrane, which is prone to bowing at higher oxygen pressures. The oxygen channels were designed to be 500 μm wide, 300 μm deep, and 17.2 cm long, resulting in a total overlap transfer area with the blood side of 0.016744 m². However, it should be understood that other ranges of channel dimensions are possible, including an open chamber instead of channels (e.g., no channels present). The layer footprint can be the same as for the blood side. Each layer of the system 100 can include a blood layer (as shown in FIGS. 1A and 1B) and an oxygen layer (as shown in FIGS. 1C and 1D) with a polydimethylsiloxane (PDMS) membrane (not pictured) sandwiched between the two. The layers can be adhered to one another, and to the membrane, using a suitable adhesive, such as an epoxy, a silicone glue, or other types of adhesives. Each of the blood layer and the oxygen layer 150 can be manufactured using the various techniques described herein, including the use of injection molding.

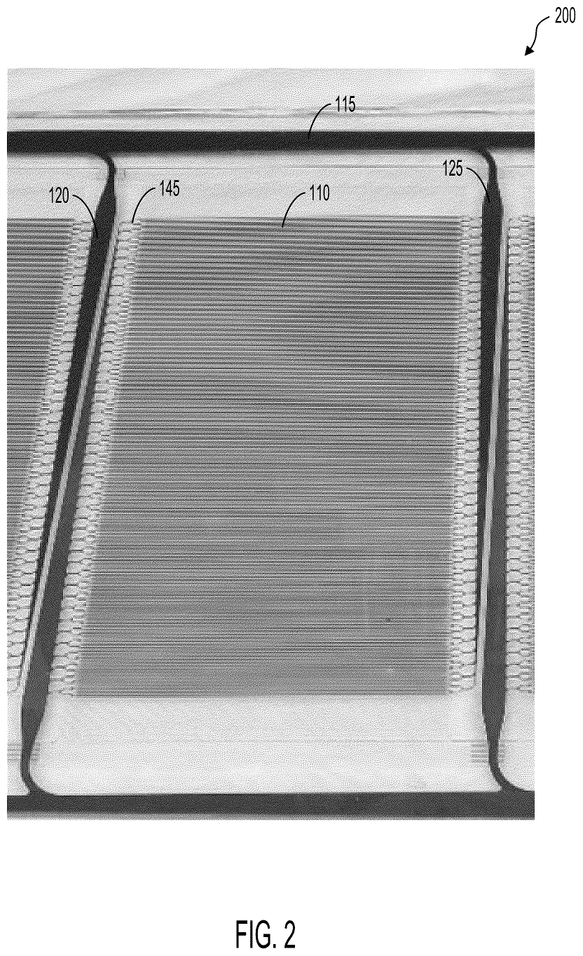

FIG. 2 is a top view 200 of photograph of a portion of an example layer similar to the blood layer shown in FIG. 1A that is oxygenating blood, according to an embodiment. As shown, the blood flows through the trunk 115, through the finger ramp 125, and into each finger 120. The blood passes through the fingers 120 and into the bifurcating channels 145, which lead to the oxygenation channels 110. As the blood passes through the oxygenation channels 110, oxygen flowing through the oxygen channels 155 of the oxygen layer 150 (not shown here) permeates through the membrane, and oxygenates the blood. The oxygenated blood then flows through another set of bifurcating channels 145, through another finger 120 and finger ramp 125, and into an outlet trunk 115, where the blood ultimately flows to an outlet. As shown, the de-oxygenated blood is dark red at the inlet, but as the blood is oxygenated, it becomes a brighter red color.

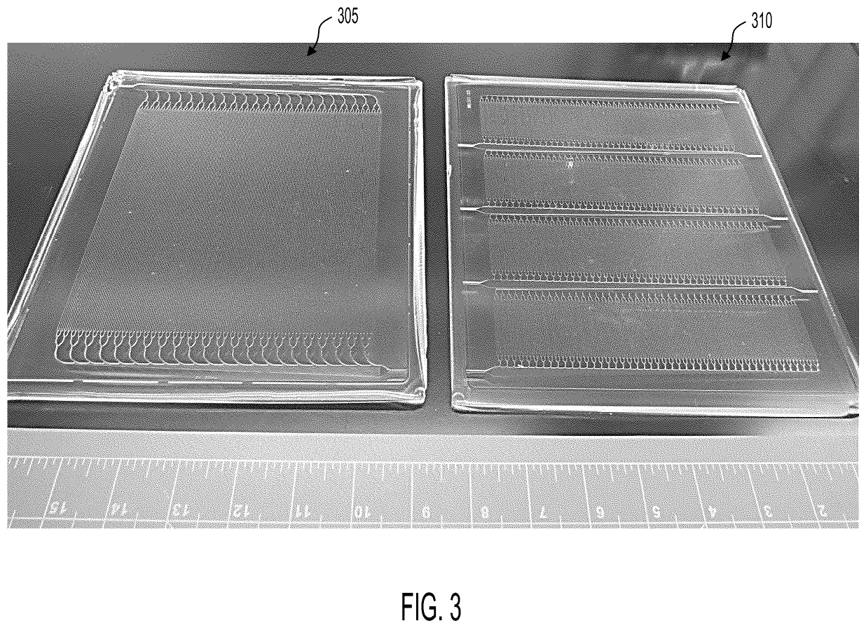

FIG. 3 is a photograph comparing a single bank design of a blood-containing layer to a multi-bank design of a blood layer that includes multiple inlets and outlets. As shown, the single-bank blood layer 305 includes just a single set of oxygenation channels, and just a single set of inlet and outlet fingers that are fluidly coupled to bifurcating channels. In contrast, the multi-bank blood layer 310 includes four banks each with a corresponding set of inlet and outlet fingers, which are fluidly coupled to trunks. In some implementations, and as shown here, the multi-bank blood layer 310 includes multiple inlets and outlets. The multi-bank blood layer 310 may be utilized in place of the blood layer shown in FIG. 1A in the system 100, for example. As shown, the oxygenation channels of the single-bank blood layer 305 are much longer than the oxygenation channels of the multi-bank blood layer 310, which is an advantage of the systems described herein.

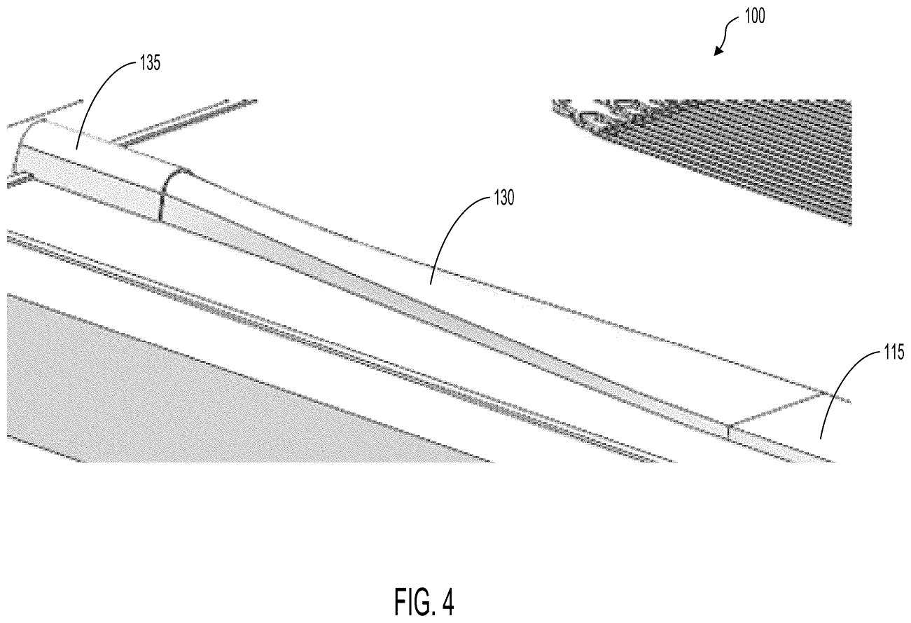

FIG. 4 is a diagram of a perspective view of an example trunk ramp 130 of the device shown in FIG. 1A, according to an embodiment. The trunk ramp 130 is created with precision geometries and a tapered transition that invoke trauma factor management. In particular, the gradual transition of the trunk ramp 130 reduces overall shear stress on the blood as the blood passes into the trunk 115 of FIG. 1A in the blood layer. As shown, the trunk ramp 130 fluidly couples the inlet 135 (or the outlet) to a corresponding trunk 115. The trunk 115 then carries the blood to or from the fingers for each bank on the blood layer, as described herein. Advantages to the design of the trunk ramp 135 are described in connection with FIGS. 5A and 5B.

FIGS. 5A and 5B are photographs showing heatmaps of the shear stress experienced by fluid when flowing through an alternative trunk ramp design and the trunk ramp of FIG. 4, respectively, according to an embodiment. As shown in FIG. 5A, an alternative design that does not implement the smooth transitions of the present techniques exhibits high shear stress on fluid in certain regions. In contrast, as shown in FIG. 5B, the trunk ramp 130, which does implement the present techniques, exhibits relatively low shear stress throughout. The design of the trunk ramp 130 improves blood health outcomes in ECMO devices even in the absence of anti-thrombogenic coatings or heavy use of anticoagulants. This approach to sizing and shaping each area of the flow path ensures the blood is exposed to a very narrow range of shear stress (e.g., 7 to 35 dynes/cm²) at nearly all points in the flow path with no sharp gradients or oscillations. The peak shear stress limit for the trunk ramp 130 was set by the desire to remain in a thrombin dominant platelet aggregation regime (e.g., less than 35 dynes/cm²) rather than a vWF regime (e.g., greater than 50 to 100 dynes/cm²). The thrombin regime allows for limited platelet aggregation with heparin-based strategies, both systemic dosage and bound surface coatings. The lower limit of 7 dynes/cm² allows for reduced material induced trauma or recirculation-like effects which exhibit as increased compliment system activation.

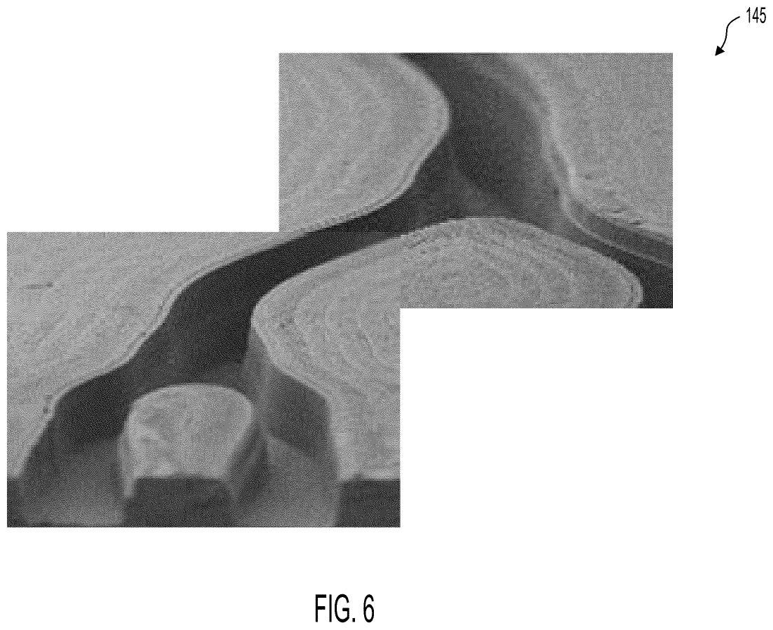

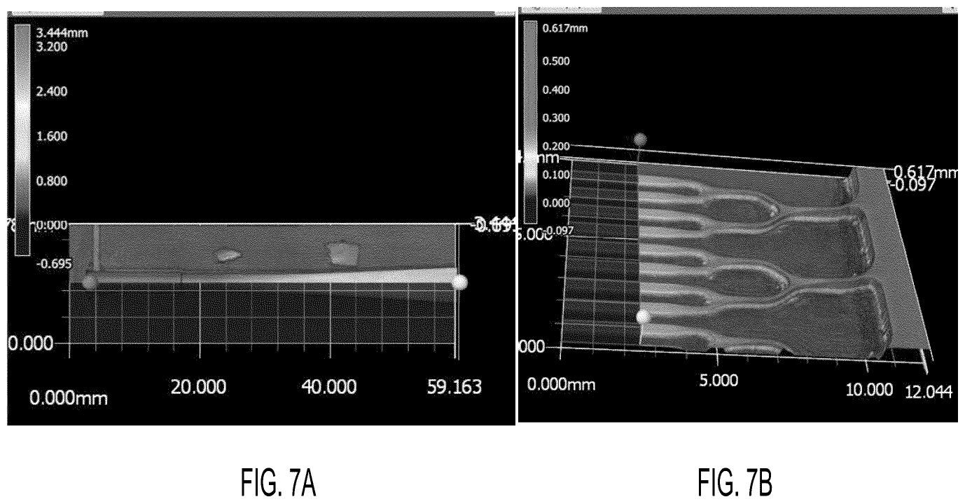

FIG. 6 is a close-up photograph of the bifurcating channels 145 (sometimes referred to as the bifurcating manifold) of the device shown in FIGS. 1A and 1B, according to an embodiment. As shown, the bifurcating channels 145 (which extend from the fingers 120 shown in FIG. 1B) divide twice, resulting in four oxygenation channels (shown in greater detail in FIG. 1B). Also as shown, the depth of the bifurcating channels decreases gradually as the bifurcating channels 145 divide. This approach to sizing and shaping each area of the flow path ensures the blood is exposed to a very narrow range of shear stress (e.g., 7 to 35 dynes/cm²) at nearly all points in the flow path with no sharp gradients or oscillations. The bifurcating channels 145 may include a geometry that provides a smooth flow path, which may include forming the channels to have smooth curves in all directions, including the depth of the bifurcating channels 145. Further details of how the depth of the bifurcating channels changes over time is shown in FIGS. 7A and 7B.

FIGS. 7A and 7B show three-dimensional (3D) metrology images of the trunk ramp 130 and the bifurcating channels, respectively, with color overlays corresponding to measured channel depth, according to an embodiment. As shown in FIG. 7A, near the outlet, the trunk ramp 310 is relatively thick (e.g., close to 3.444 mm). The trunk ramp 130 flattens out moving towards where the trunk ramp 130 connects to the trunk 115, resulting in a gradual transition to a lower channel depth. Similar changes to channel depth are shown in the models of the bifurcating channels in FIG. 7B, which shows the channel depth of the bifurcating channels reduces as the channels extend from the finger 120.

FIGS. 8A, 8B, and 8C show example views of a device similar to that shown in FIG. 1A with example membrane support features in the oxygen-carrying layer, according to an embodiment. FIG. 8A shows a top photograph of 800A a device similar to that shown in FIG. 1A, and FIG. 8B shows a detailed block diagram 800B of the photograph in FIG. 8A. FIG. 8C shows a perspective view 800C showing the support features 810 in the oxygen trunk 805 of the oxygen layer. Oxygen compartment supports 810 in certain locations of the oxygen layers can prevent membrane buckling under oxygen backpressure. The membranes described herein may be manufactured from PDMS. Due to the elastic nature of PDMS, the thin oxygen transfer membrane can be deflected by sufficiently large pressure difference across the membrane. Pressure differences are unavoidable in normal operation of the device, it is important to minimize the degree of deflection in order to maintain the flow geometry in the oxygen channels 815. This is accomplished by minimizing the unsupported span of the membrane in regions where the blood and oxygen channels 815 cross. These channels primarily cross over in the small oxygen transfer channels 815 where the unsupported span can be kept around 250 μm due to the small size of the channels. However, in areas where the larger distribution channels necessarily must cross, this is more problematic.

In order to minimize deflection in these areas, small raised support structures 810 can be added to the oxygen side flow path 805 to keep the largest span to 250 μm, which dramatically decreases the channel deformation in these areas. The support structures 810 may be defined as part of the oxygen flow path 805 (e.g., in a mold during manufacturing), or may be added after manufacturing. Some examples of support structures include foam, solid structures, or other types of structures that enable the flow of oxygen while being rigid enough to support the membrane while under pressure. Additionally, the width of the oxygen channels can be reduced in order to limit the unsupported membrane span which results in significantly less centerline deflection for the same applied pressure. The oxygen layer may be defined such that oxygen channels do not cross over the finger portions of the blood side of the device.

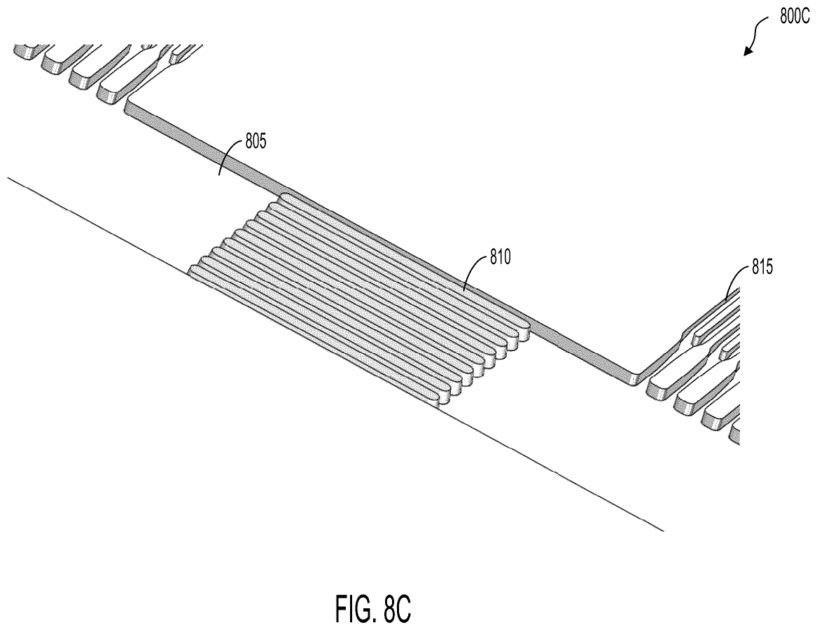

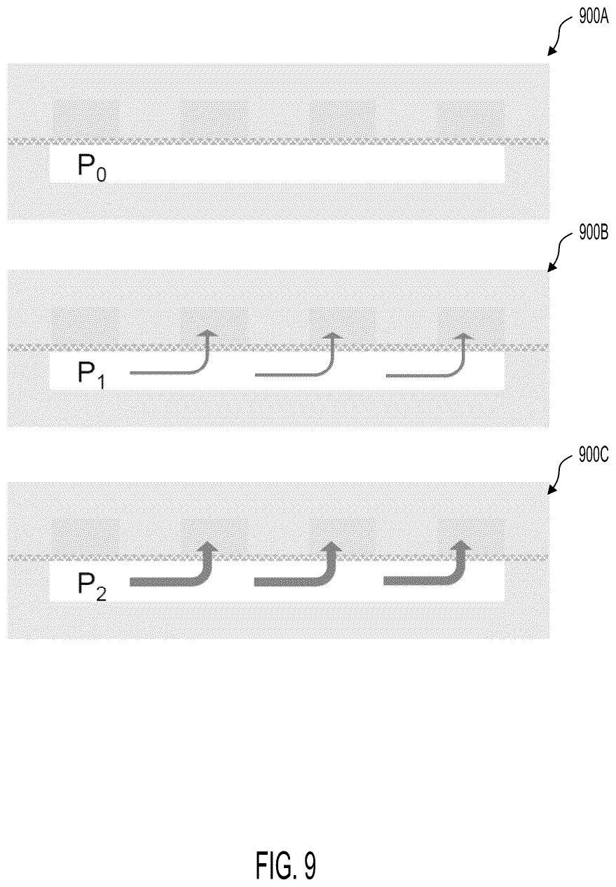

FIG. 9 shows example block diagrams 900A, 900B, and 900C showing the application of different oxygen backpressures on a membrane in a device similar to that shown in FIG. 1A, according to an embodiment. In the diagram 900A, static flow conditions are shown with no pressure exerted in either channel. In the example shown in the diagram 900A, the pressure P₀ is equal to 0 mmHg. In the diagram 900B, sweep gas flow rates facilitate gas transfer across the membrane, and exert minimal pressure on the gas channel. In the example shown in the diagram 900B, the pressure P₁ is about equal to a range of 4 mmHg to 50 mmHg. In the diagram 900C, applied backpressure in the gas channel drives bulk movement of gas (e.g., oxygen) across the membrane. In the example shown in the diagram 900C, the pressure P₂ is about equal to a range of 50 mmHg to 500 mmHg.

FIG. 10 shows a diagram of an example system 1000 in which a pressure controller can apply oxygen backpressure in a device similar to that shown in FIG. 1A to improve oxygenation of blood flowing through the device, according to an embodiment. As shown, the system 1000 includes a device similar to that described in connection with FIGS. 1A-1D, the oxygen layer of which receives oxygen from an oxygen tank controlled by an oxygen flow controller and a pressure sensor. In this example, a syringe pump provides blood to the blood layer of the device, which flows to an output and can be tested using an avoximeter or a blood analyzer. Additionally, the system 1000 includes a dual-valve pressure controller at the outlet of the oxygen layer. The dual valve pressure controller can apply oxygen backpressure at the outlet to achieve a predetermined uniform pressure throughout the device. Any suitable device can be utilized to apply oxygen backpressure.

The application of oxygen backpressure can increase oxygen transfer efficiency. Current extracorporeal oxygenator technology operates with a sweep gas flow rate that is associated with a pressure drop in the gas channel. In microfluidic devices, this pressure drop is proportional to the volumetric flow rate of the gas and may be a driving factor of gas transfer across the membrane and into/out of the blood. To improve oxygen transfer efficiency, increasing the bulk movement of oxygen molecules across the membrane can be achieved by applying a user-defined backpressure to the oxygen chamber. This can be done, for example, by using a dual-valve pressure controller with access to house air and vacuum, or another suitable pressure-controlling device. The increase of convective transport of oxygen into the blood channel is achieved without changing blood flow rates or channel geometries to improve transfer efficiencies. Applied oxygen backpressures have been tested between 0-400 mmHg in in vitro tests. In all configurations tested, a backpressure as low as 50 mmHg has been enough to increase oxygen transfer at a given blood flow rate.

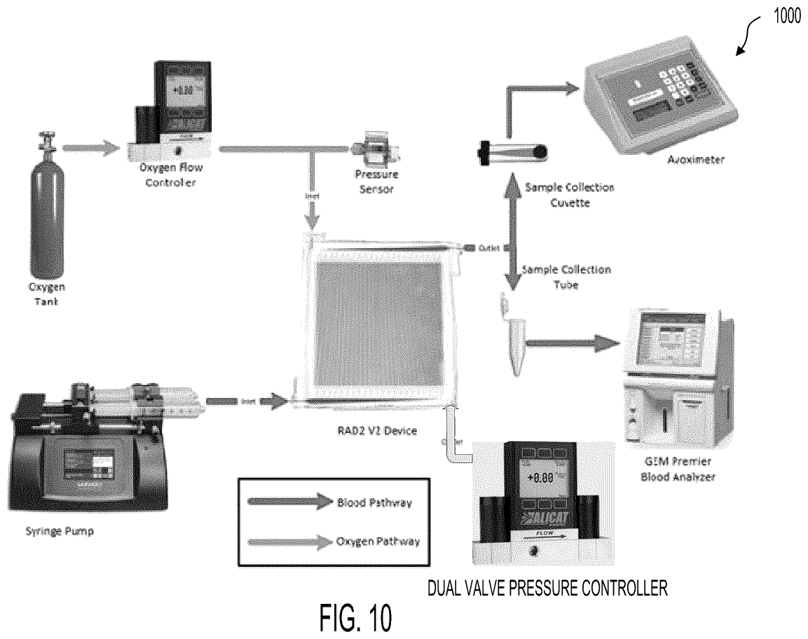

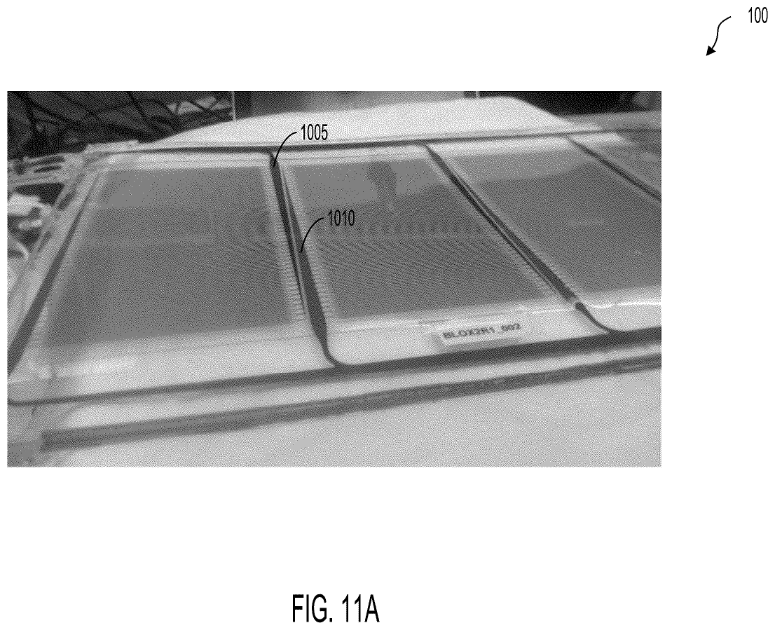

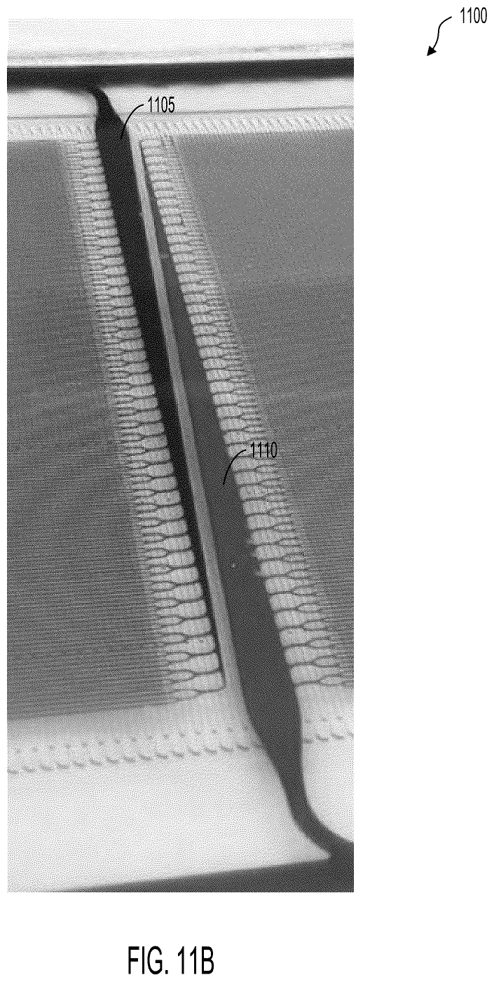

FIGS. 11A and 11B show perspective and zoomed views, respectively, of an example device similar to that shown in FIG. 1A that is oxygenating blood, according to an embodiment. As shown in FIG. 11A, and in the zoomed view 1100 of FIG. 11B, the inlet finger 1005 and the outlet finger 1010 (which may be similar to the fingers 120 described herein) are each saturated with blood. The inlet finger 1005 is saturated with de-oxygenated blood, and therefore has a darker color. Once the blood has been oxygenated in the oxygenation channels of the system 100, the oxygenated blood passes into the outlet finger 1010. As shown, the outlet finger 1010 is saturated with oxygenated blood, and therefore has a bright red color.

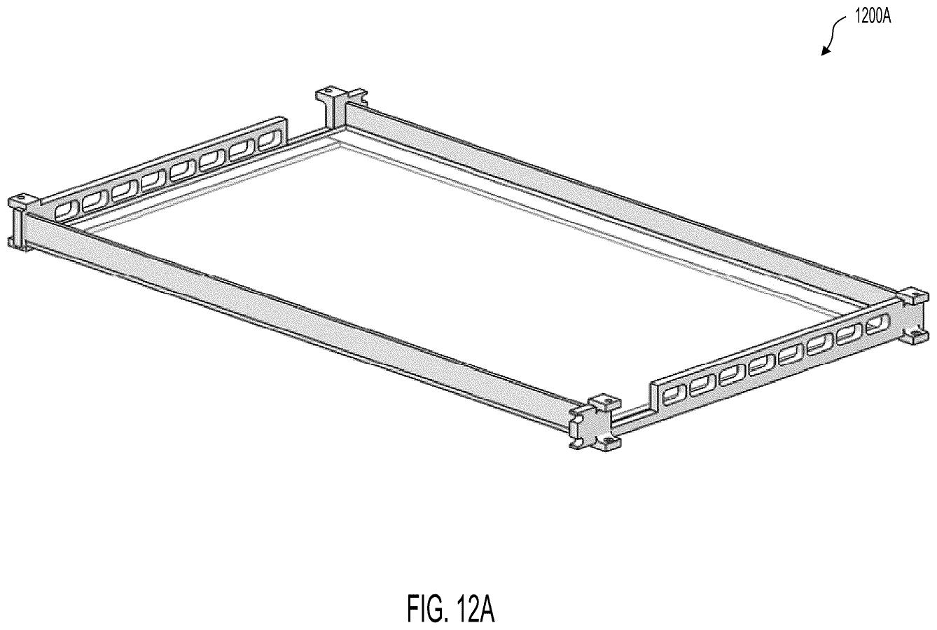

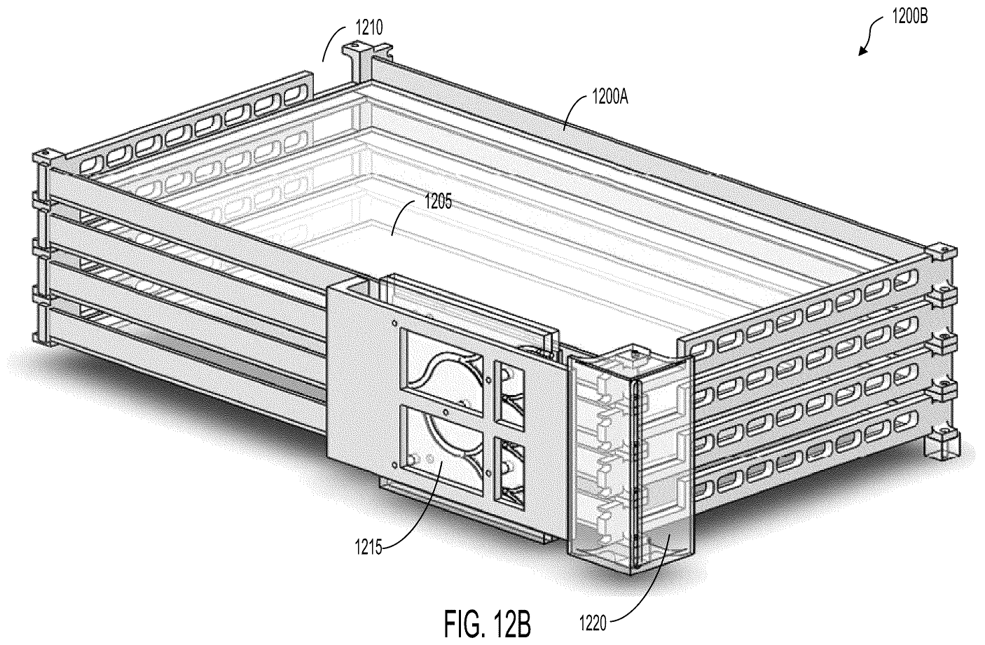

FIGS. 12A and 12B show an example tray 1200A and housing 1200B, respectively, which may be utilized to house one or more layers similar to of the system 100 described in connection with FIGS. 1A-1D, according to an embodiment. As shown, the housing 1200B includes a number of trays 1200A. Each tray 1200A can include a clear plastic bottom 1205, as well as a clearance region 1210 for blood and oxygen access. Each tray 1200A can include one or more connectors or features that enable the tray 1200A to couple with other trays 1200A. A vertical manifold 1215 can be used to uniformly distribute blood to each layer, as described herein. A protective casing 1220 can be used to protect tubing that extends from the manifold and into the clearance regions 1210. Although four trays 1200A are shown here, it should be understood that any number of trays (with suitable vertical manifold(s) 1215) may be utilized to increase the layer count of the device. Each tray 1200A can house a single oxygenator layer (e.g., a layer 100 and a layer 150 with a membrane disposed in between as described herein).

The housing 1200B can support and stabilize the layers of the oxygenator. The housing 1200B includes a stackable tray 1200A system that improves the assembly, stability, and portability of the multi-layer stack devices 100. Each tray can support 1-2 layers (e.g., a single layer of an ECMO device such as the system 100) and provides mechanical support to the otherwise flexible layers. Tubing connections to each layer 1200A are protected by the casing 1220, which prevents accidental damage during device operation. Additional manifolds 1215 for connecting the layers is integrated with the stacked trays and improves usability.

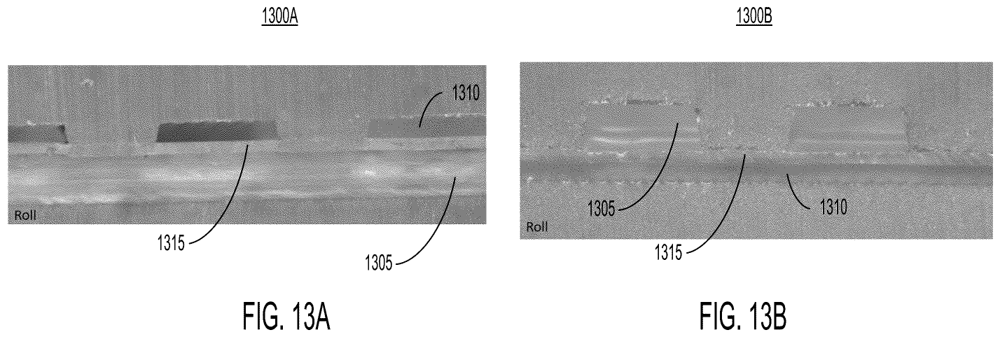

FIGS. 13A and 13B show cross-sectional photographs 1300A and 1300B, respectively, of a single-membrane design for a device similar to that shown in FIG. 1A, according to an embodiment. The photograph 1300A shows a membrane 1315 (e.g., a PDMS membrane as described herein) sandwiched between a blood layer including one or more blood channels 1310 (e.g., similar to the oxygenation channels 110 of FIGS. 1A and 1B) and a perpendicular oxygen channel 1305 (e.g., similar to the oxygen channel 155 of FIGS. 1C and 1D). The photograph 1300B shows the same material but at a perpendicular cross-section (e.g., facing the oxygen channels 1305 and parallel to a blood channel 1310).

The membrane 1315 can be integrated with the layers of the device using an example two-step bonding process. The two-step bonding process can integrate a pre-casted sheet or rolled membrane 1310 into the multi-banked microfluidic oxygenator device (e.g., one or more layers of the system 100). One example of a rolled membrane 1310 is a cross-linked silicone rubber film, designed to be biocompatible for the healthcare industry. In this example, the membrane has a width of 250 mm and a range of thickness between 20 to 400 μm. The film can be first bonded to the oxygen layer (e.g., the oxygen layer 150) and cured under the application of weight for at least 48 hours at room temperature. This initial bond step to the oxygen layer is important because the release of the bonded membrane from the film backer can cause unwanted tension in the channel areas that span large gaps. By doing the oxygen bond first, the possible tension and subsequent release does not have an impact in the blood layer. The second bond is to the blood layer, completing the device assembly. Both rolled/sheet membranes and cast membranes can be utilized.

FIGS. 14A, 14B, and 14C show photographs 1400A, 1400B, and 1400C of an example vertical manifold that may be utilized with multiple parallel layers of the device shown in FIG. 1A, according to an embodiment. The vertical manifolds shown in the photographs 1400A, 1400B, and 1400C can be cast from a suitable material, such as silicone, to enable layer-to-layer integration. The vertical manifolds may be integrated with a housing (e.g., the housing 1200B described in connection with FIGS. 12A and 12B).

Vertical manifolds were designed and fabricated to equally distribute blood in and out of a multi-layer stack of device layers (e.g., the system 100 described in connection with FIGS. 1A-1D). The in-layer manifold connecting the multi-bank design can coalesce into a single inlet and outlet for each microfluidic device layer. In an 8-layer stack, for example, each of these inlets and outlets are merged by interfacing with a branched manifold that resides external and vertical to the stack. Even blood distribution to the stack is important and is achieved by an equal resistance branching design of the manifold. The manifold fabrication can be accomplished using a replica molding approach, comprised of a machined master mold and casted silicone elastomer. Alternative manufacturing techniques are also possible, such as 3D printing, machining, or other approaches.

Precise placement of the vertical manifolds can be based on alignment features that are present through the replica casting through the final assembly of the stack. Threaded through-holes can be utilized for placements for aluminum alignment pegs. These pegs can result in negative space, and inverse through-holes in the ultimate casted halves of the silicone elastomer. Two casts are aligned using these features and bonded using an RTV adhesive. The final manifold assembly is placed along the edge of the stack and interfaces with the inlet/outlet of individual device layers (e.g., as shown in FIG. 12B). In one iteration, the manifold splits a ¼″ inner-diameter tubing-compatible channel to 8⅛″ inner-diameter tubing-compatible channels, which can be used for both an inlet and outlet manifold. The vertical manifold can have a symmetrical bifurcated design, as shown in the photograph 1400A.

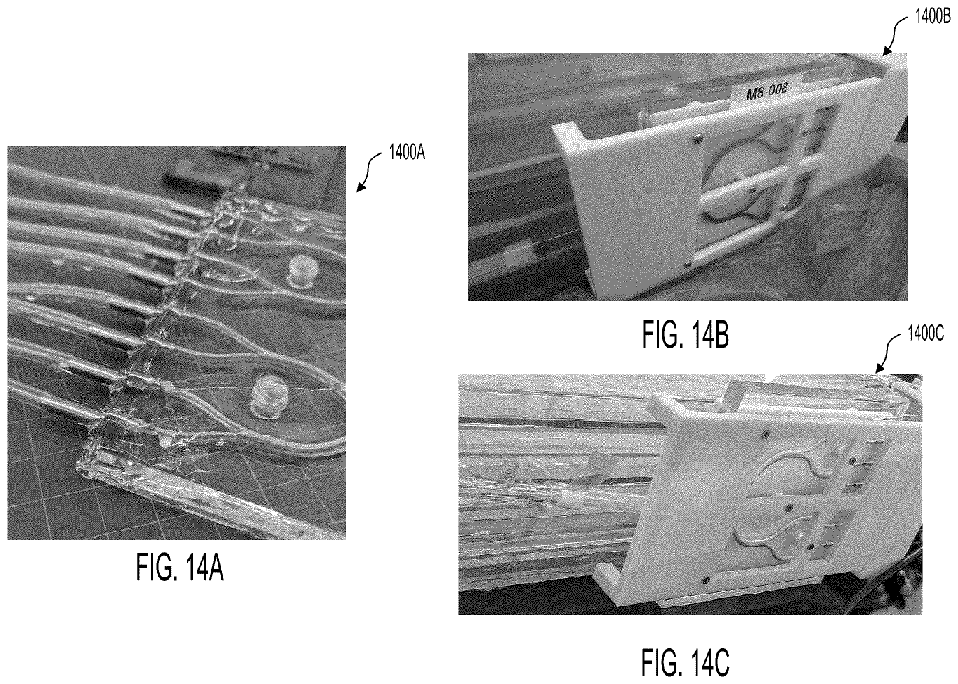

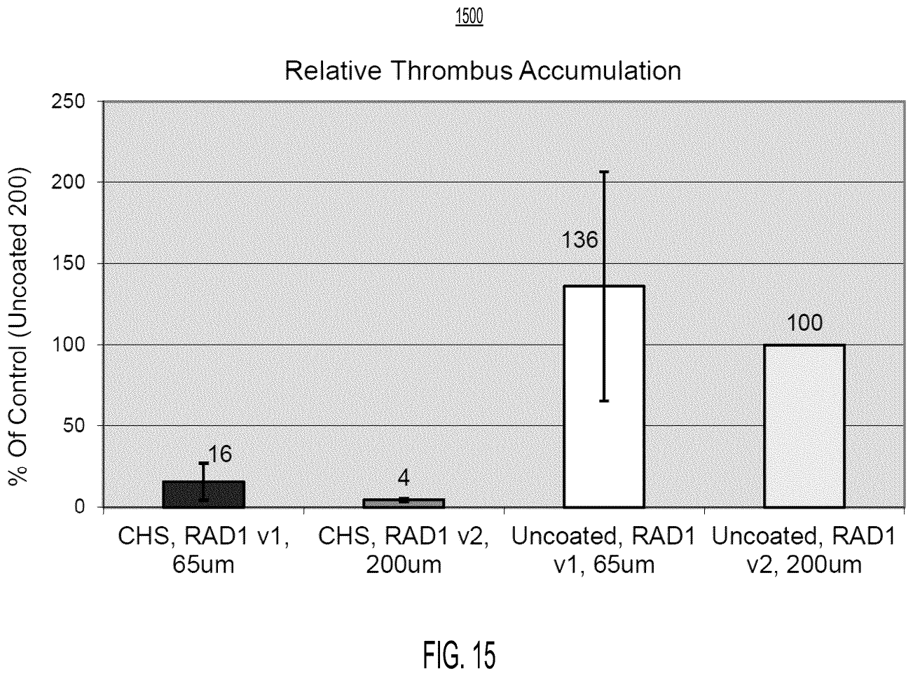

FIG. 15 provides a graph 1500 showing thrombus accumulation relative to a control for coated and uncoated devices, according to an embodiment. Various coatings can improve the hemocompatibility of the blood layer to further reduce the shear stress experienced by the blood traveling therethrough. The bio-inspired design of the microfluidic channel geometries in the blood layer shown in FIGS. 1A and 1B support blood health the system 100 by reducing the range of shear stress acting on the blood as it travel through the channels 110. The system 100 is also compatible with surface coating technology, which comes in many forms, to further maintain blood health during the device operation. Surface coating technologies include heparin-based coatings like CHS (Corline heparin solution, Corline Biomedical), non-adhesive technologies like tethered liquid perfluorocarbon (TLP) or infusions of silicone oil, and nitric oxide donors. Some of these coatings, like the CHS, can be introduced into the channels and incubated before being flushed out. As shown in the graph 1500, CHS can be shown to reduce increases in blood pressure drop over time and reduce platelet counts compared to uncoated channels.

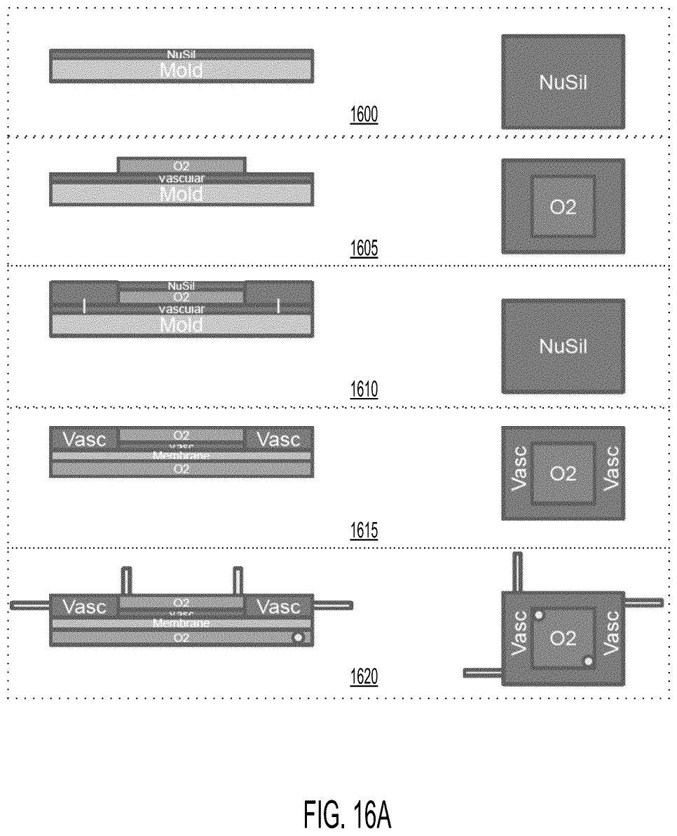

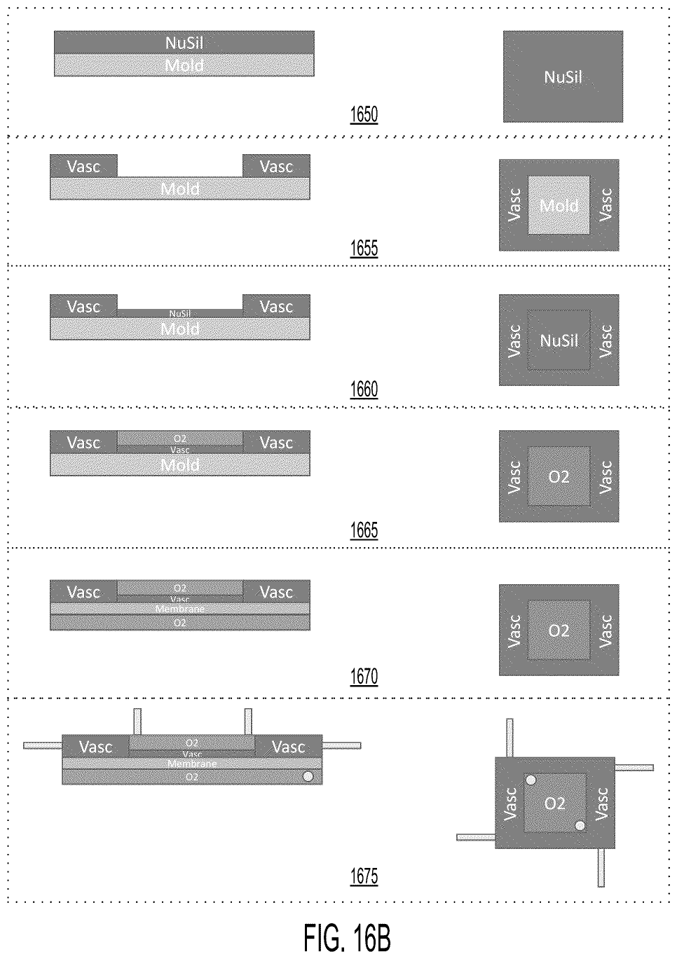

FIG. 16A shows a diagram illustrating a process for manufacturing an example dual-membrane device that is similar to the single-membrane devices described herein, according to an embodiment. Dual-membrane configurations of microfluidic oxygenators (e.g., a variation of the system 100 described in connection with FIGS. 1A-1D) can enable oxygen transfer from two planes of the blood channel, rather than one. In a multi-banked design, a construction process can be utilized to account for both the distribution channels and entrance/exit regions of the blood layer. One of the two membranes can be formed by nominal fabrication procedure using the membrane roll. This membrane can be 50 μm thick. The second membrane can be created by a series of masking, casting and aligning tubing interfaces. The second membrane can be up to 200 μm thick, but may have a different thickness or may vary in thickness.

At step 1600, a thin layer of a silicone material (e.g., NuSil, etc.) can be poured and cured in a mold, as shown, to form a blood layer. The thickness of the blood layer can be any suitable thickness, as described herein, to cover the channels plus a height of a membrane. The blood layer can include features similar to those depicted in FIGS. 1A and 1B. Once the silicone material has been cured, at step 1605, a pre-cured and trimmed oxygen layer can be bonded to the layer of silicone where the oxygen channels would cross the blood channels (e.g., perpendicularly, as described herein). The oxygen layer can be formed using the techniques described herein, and may include features similar to those depicted in FIGS. 1C and 1D.

At step 1610, additional silicone (e.g., NuSil) can be poured into the mold to create the trunk geometry and to seal in the oxygen layer, as shown. The additional silicone can be cured using suitable curing techniques, and then the complex of cured layers can be removed from the model for further process steps. At step 1615, a second oxygen layer, with a membrane bonded thereto, can be bonded to the previously cured blood layer formed in step 1610. As shown, one side of the membrane is bound to one side of the second oxygen layer, and the other side is bonded to the blood layer. The membrane can be PDMS material, as described herein. The second oxygen layer can be formed using techniques similar to those for the first oxygen layer, and may include various features shown in FIGS. 1C and 1D. Then, at step 1620, inlet and outlet ports can be provided at appropriate regions, and the edges of the device can be sealed using silicone, an adhesive, or another suitable solution. The materials in this process can be bonded to one another using a silicon adhesive, an epoxy resin, or another type of glue. In an embodiment, thermal bonding may be utilized to bond two or more layers to one another. As shown, the inlets and outlets of the top oxygen layer extend perpendicular to the stack, while the other inlets and outlets for the blood layer and the second oxygen layer extend parallel to the stack.

FIG. 16B shows a diagram illustrating another process for manufacturing an example dual-membrane device that is similar to the single-membrane devices described herein, according to an embodiment. As described herein, Dual-membrane configurations of microfluidic oxygenators (e.g., a variation of the system 100 described in connection with FIGS. 1A-1D) can enable oxygen transfer from two planes of the blood channel, rather than one. Some of the process steps described in connection with FIG. 16B may be similar to those described in connection with FIG. 16A.

At step 1650, a layer of a silicone material (e.g., NuSil, etc.) can be poured and cured in a mold, as shown, to form a blood layer. The blood layer can include features similar to those depicted in FIGS. 1A and 1B. Once the silicone material has been cured, at step 1655, an active area (e.g., corresponding to the location where the oxygen channels would cross the blood channels) can be removed from the cured layer of silicone material. The material can be removed using any suitable process (e.g., cutting, chemical removal, etc.). When the material is removed, the silicone making up the trunk geometry of the blood layer can remain. At step 1660, additional silicone material can be poured into the cut out area to create a thin silicone membrane above the channels. The thin membrane can have dimensions similar to those described in connection with FIGS. 18A and 18B, for example.

Once the thin membrane silicone has cured, at step 1665, a pre-cured and trimmed oxygen layer can be bonded to the silicone membrane in the remaining active region (e.g., where the oxygen channels would perpendicularly cross the blood channels). The oxygen layer can be formed using the techniques described herein, and may include features similar to those depicted in FIGS. 1C and 1D. Once the oxygen layer has been bonded to the silicone membrane, additional silicone material can be deposited to seal all seams and create a continuous layer. The additional silicone can then be cured. At step 1670, a second oxygen layer, with a membrane bonded thereto, can be bonded to the previously cured blood layer formed in step 1610. As shown, one side of the membrane is bound to one side of the second oxygen layer, and the other side is bonded to the blood layer. The membrane can be PDMS material, as described herein. The second oxygen layer can be formed using techniques similar to those for the first oxygen layer, and may include various features shown in FIGS. 1C and 1D.

At step 1675, inlet and outlet ports can be provided at appropriate locations on the dual-membrane device, and the edges of the device can be sealed using silicone, an adhesive, or another suitable solution. The materials in this process can be bonded to one another using a silicon adhesive, an epoxy resin, or another type of glue. In an embodiment, thermal bonding may be utilized to bond two or more layers to one another. As shown, the inlets and outlets of the top oxygen layer extend perpendicular to the stack, while the other inlets and outlets for the blood layer and the second oxygen layer extend parallel to the stack. The inlets and outlets can be formed of any suitable material, such as a hemocompatible plastic material.

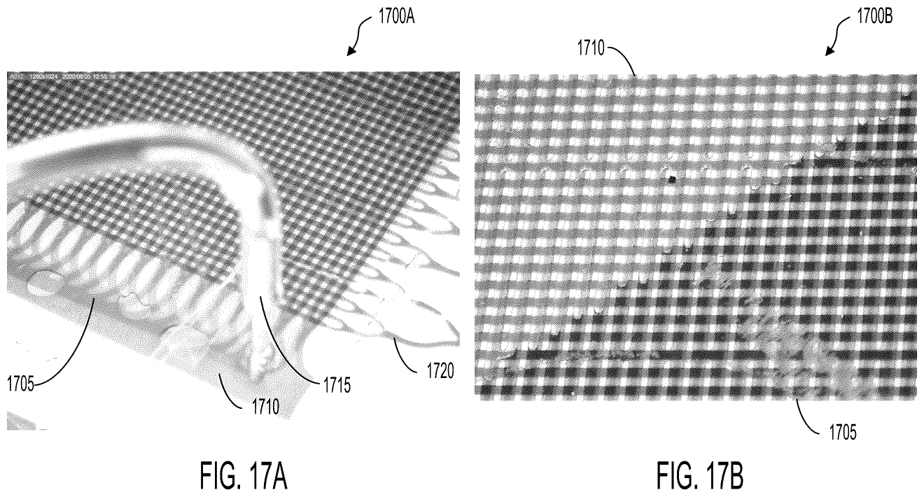

FIGS. 17A and 17B show photographs 1700A and 1700B of an example dual-membrane device with two oxygen-carrying layers, according to an embodiment. The dual-membrane device can be manufactured based on the techniques described in connection with FIG. 16A or 16B. The dual-membrane device can have three transport layers, two oxygen layers 1705 and 1710, and one blood layer 1720. The blood layer 1720 may be similar to the blood layer described in connection with FIGS. 1A and 1B, and the oxygen layers 1705 and 1710 may be similar to the oxygen layer described in connection with FIGS. 1C and 1D. As shown in the photograph 1700A, a top inlet 1715 can provide oxygen to the oxygen channels of the top oxygen layer 1705. Although not shown here, the blood layer can be separated from each of the top oxygen layer 1705 and the bottom oxygen layer 1710 by two respective membranes. The photograph 1700B shows a close-up view of how the bottom oxygen layer 1710 and the top oxygen layer 1705 overlap each side of the blood layer 1720.

FIGS. 18A and 18B show cross-sectional photographs 1800A and 1800B of a dual-membrane as described herein, according to an embodiment. The photograph 1800A shows a blood layer 1810 (e.g., including blood channels similar to the oxygenation channels 110 of FIGS. 1A and 1B) sandwiched between two membranes 1815A and 1815B (e.g., which may be PDMS membranes as described herein). On the other side of each membrane are respective oxygen layers 1805A and 1805B (e.g., which can include oxygen channels similar to the oxygen channels 155 of FIGS. 1C and 1D). As shown in the photograph 1800A, the membrane 1815B can be thicker than the membrane 1815A. Like the single-membrane design, the channels of the blood layer 1810 can be perpendicular the oxygen channels of the oxygen layers 1805A and 1805B. The photograph 1300B shows the same material but at a perpendicular cross-section (e.g., facing the blood channels of the blood layer 1810 and parallel to the oxygen channels 1805A and 1805B). The membranes 1815A and 1815B can be integrated with the layers of the device using the techniques described in connection with FIG. 16A or 16B.

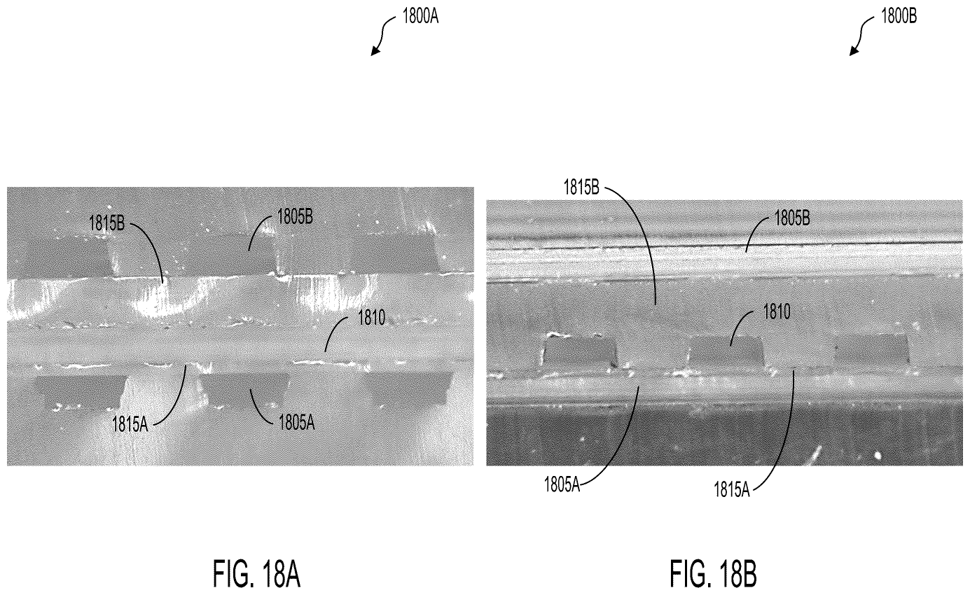

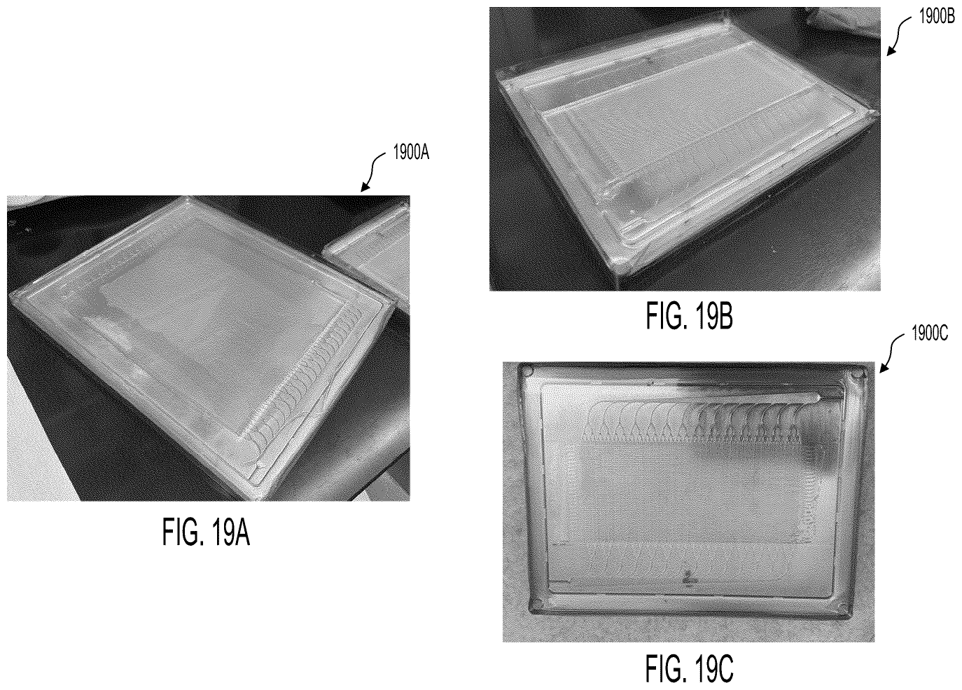

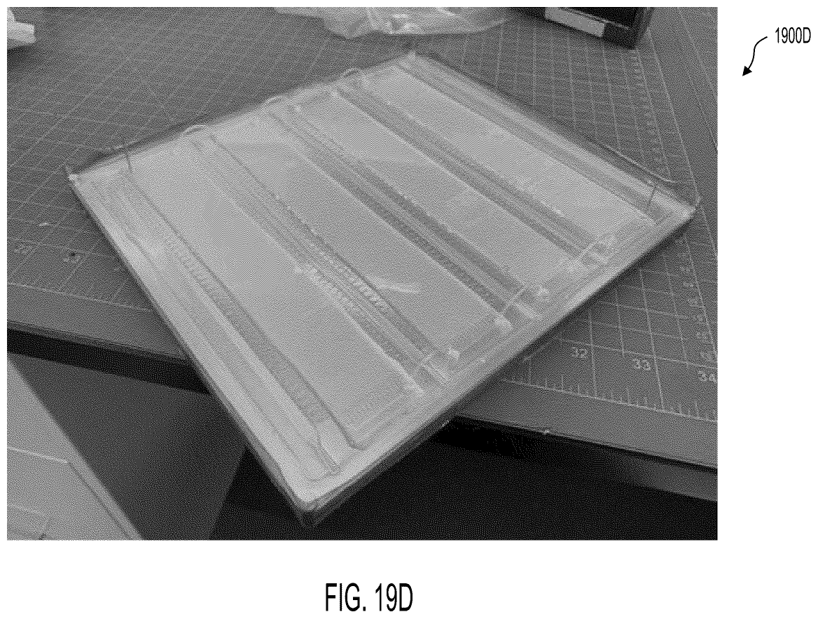

FIGS. 19A, 19B, 19C, and 19D show example photographs 1900A, 1900B, 1900C, and 1900D of an example manufacturing process for the dual-membrane devices described herein (e.g., similar to those described in connection with FIGS. 16A-18B), according to an embodiment. The manufacturing processes used to fabricate the dual-membrane devices may be similar to the processes described in connection with FIG. 16A or 16B. The photograph 1900A shows an example stage in a process for forming a dual-layer device, as described in connection with FIG. 16B. The photograph 1900A shows an example stage in which a thin blood layer is formed on a mold such that the top surface of that layer serves as a membrane. The photographs 1900B and 1900C shows an example stages in a process similar to that described in connection with FIG. 16A. The photograph 1900D shows a similar process stage for manufacturing a dual-membrane device.

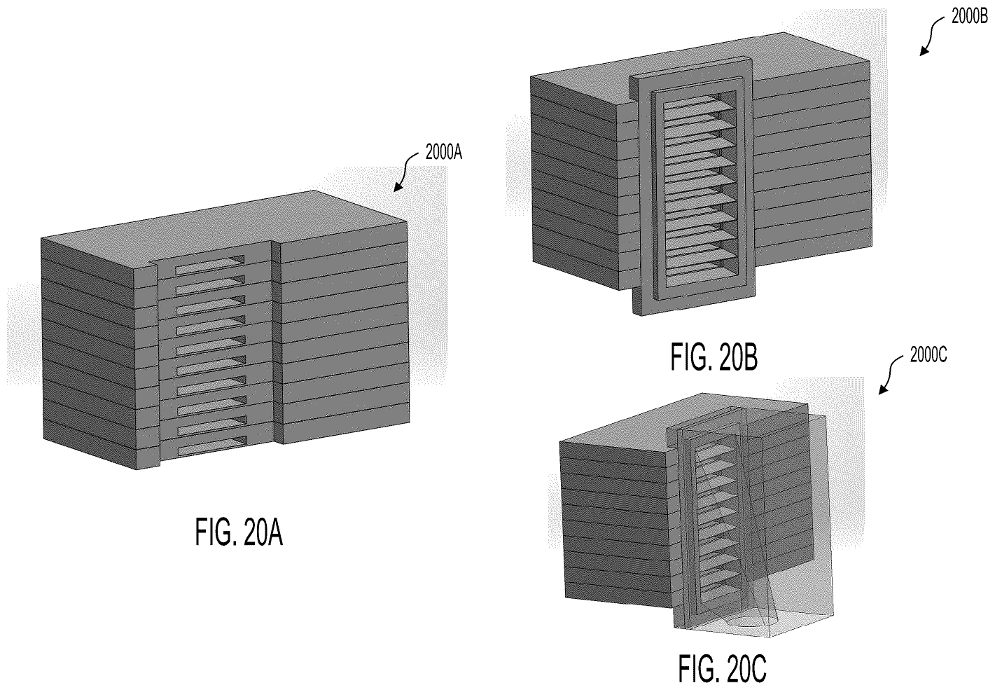

FIGS. 20A, 20B, and 20C show perspective diagrams 2000A, 2000B, and 2000C of an example edge manifold with rectangular ducts, according to an embodiment. The edge manifold shown in the diagrams 2000A, 2000B, and 2000C can be an alternative edge manifold design that can eliminate “mouse hold” tubing access. As shown in the diagram 2000A, the edge manifold can include entrance and exit channels that terminate at the layer edge (e.g., an edge of the blood layer shown in FIG. 1A or the oxygen layer shown in FIG. 1C, etc.) and include rectangular ducts instead of circular ducts. Each of the ducts can have a predetermined thickness (e.g., 2.5 mm per layer), and can stack on top of one another to correspond to the number of layers in the device. Each duct layer can be manufactured using a suitable technique, such as injection molding, 3D printing, or machining, among other techniques. The layers can be secured to one another using a suitable adhesive, such as an epoxy resin, a silicone glue, or another type of glue.

In an embodiment, the layers to which the edge manifold is connected can be formed to have rectangular inlets and outlets to conform to the rectangular ducts. Eliminating the round tuning connections to each layer can allow the layers to be much thinner overall, which can greatly reduce the overall device size and weight, particularly when many layers are utilized in a single device. As shown in the diagram 2000B of FIG. 20B, a frame can be placed over the stack of rectangular ducts to aid with alignment/attachment of a single plenum. As shown in the diagram 2000C of FIG. 20C, a molded, single large plenum can be affixed to the layer stack over the entrance/exit ducts that distribute the blood uniformly to every layer. The plenum can provide a single access point for blood entering or exiting the plenum to or from the pump or patient.

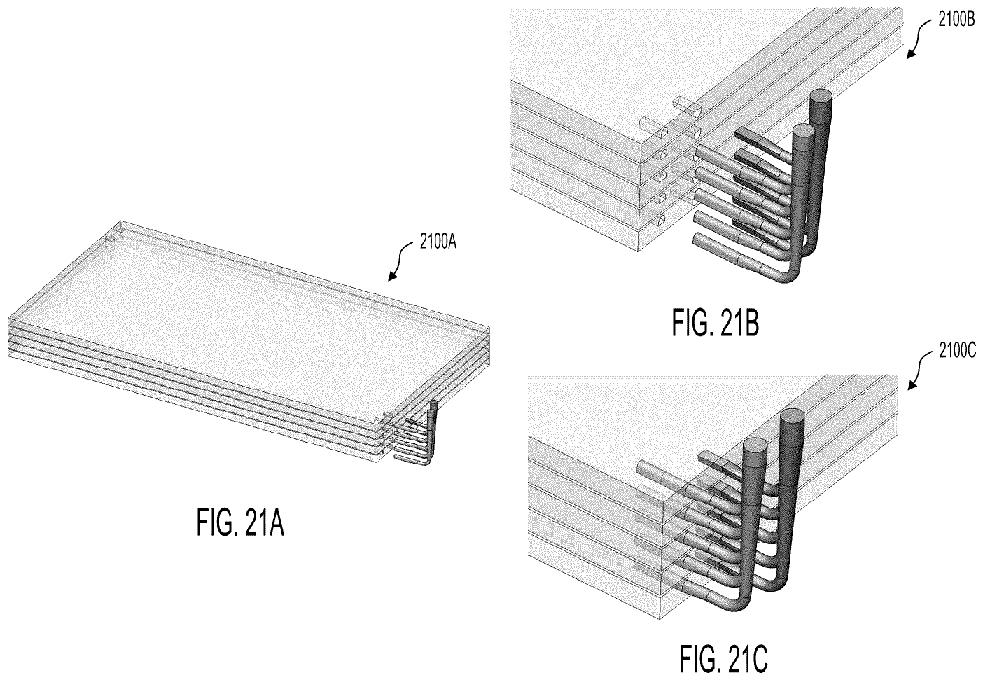

FIGS. 21A, 21B, and 21C show perspective diagrams 2100A, 2100B, and 2100C of interior space constructs used in a manufacturing process for an alternative edge manifold, according to an embodiment. The alternative edge manifold may sometimes be referred to as a “lost wax” manifold. Making connections between flat fluidic device layers (e.g., the device layers described in connection with FIGS. 1A-1D) can be used to increase processing capacity by running the devices in parallel. In these devices, the transitions for the blood passages must be smooth to avoid undesirable fluidic behavior that cause blood damage and clotting. The alternative lost wax edge manifold approach to connecting multiple flat device layers. This approach can produce nearly seamless and smooth channel walls, and can accommodate variability between device thickness and geometry.

The layers for the blood oxygenation device, as described herein, can include a blood layer and an oxygen layer, separated by a membrane. For simplicity, each bilayer component is represented here as a large flat rectangle with only the ports depicted. In the process to manufacture the alternative edge manifold, the interior negative space of the manifold is fabricated using a soft and flexible material, which can also be melted or dissolved in subsequent process steps. One example material can be a wax material that is cased in a 2-part mold (e.g., which itself may be machined using high-resolution CNC machine). Another approach to forming the interior negative space construct of the manifold can include 3D printing the construct. The interior negative space of the manifold will be used to cast the manifold itself, and will then be subsequently removed. The fabrication process for the interior negative space portions can be of a high resolution, such that the surfaces are smooth. Smooth surfaces will define smooth surfaces in the manifold, once case, to minimize shear stress experienced by blood cells during device operation.

Once the interior space constructs have been case, the device layers can be stacked. In an embodiment, spacers can be placed between some of the layers to make the spacing regular. The manifold interior-space constructs can then be inserted into the ports of the device layers, as shown in the diagrams 2100A, 2100B, and 2100C. Red can represent the construct for the blood layer ports, and blue can represent the construct for the oxygen layer ports. As shown, the geometry of the constructs can be shaped to match the ports, such that the fit is snug. Additionally, since the constructs are flexible they can bend to accommodate variations in inter-layer port spacing.

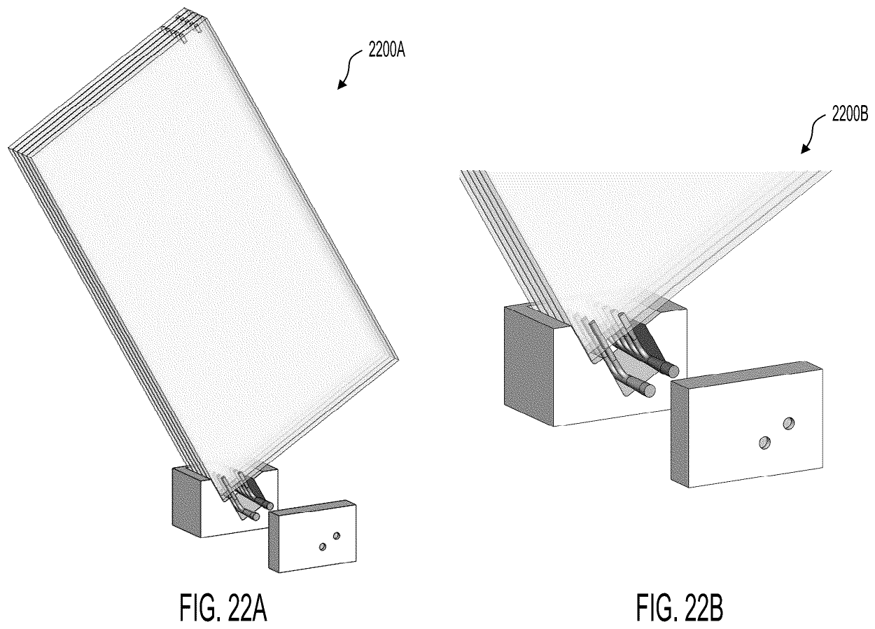

FIGS. 22A and 22B show example views 2200A and 2200B of an example process for manufacturing the alternative edge manifold using the interior space constructs shown in FIGS. 21A, 21B, and 21C, according to an embodiment. As shown, after inserting the interior constructs into the ports of the layers, the layers and the constructs can be placed inside a mold for the alternative edge manifold. The manifold negative space constructs ports can pass through holes on one side of the mold. FIGS. 23A and 23B show additional example views 2300A and 2300B of the example process for manufacturing the alternative edge manifold using the interior space constructs shown in FIGS. 21A, 21B, and 21C, according to an embodiment. After inserting the corner of the device with the interior constructs into the mold, the mold can be sealed, as shown in the view 2300A of FIG. 23A. A cross-sectional view of the mold is shown in 2300B, which depicts how the interior constructs are suspended in the middle of the mold by the holes on the cover of the mold and by the ports of the device.

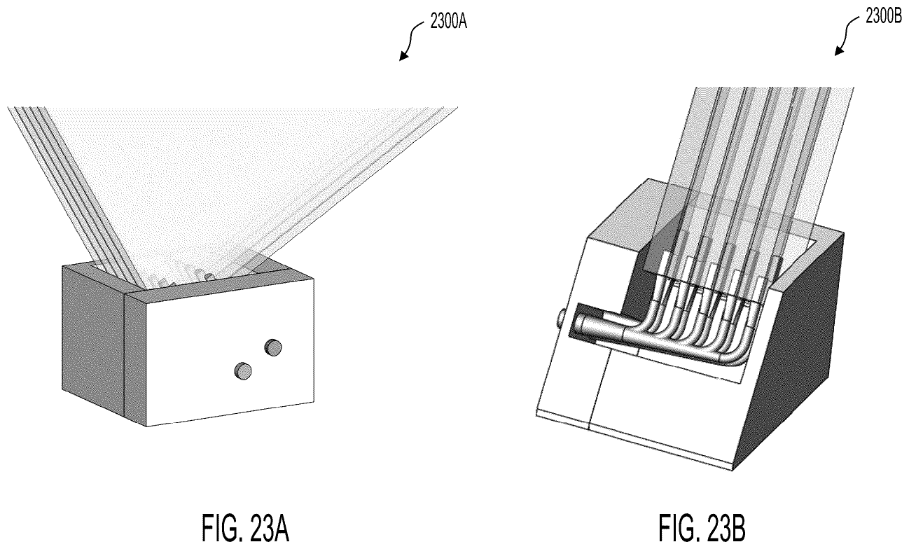

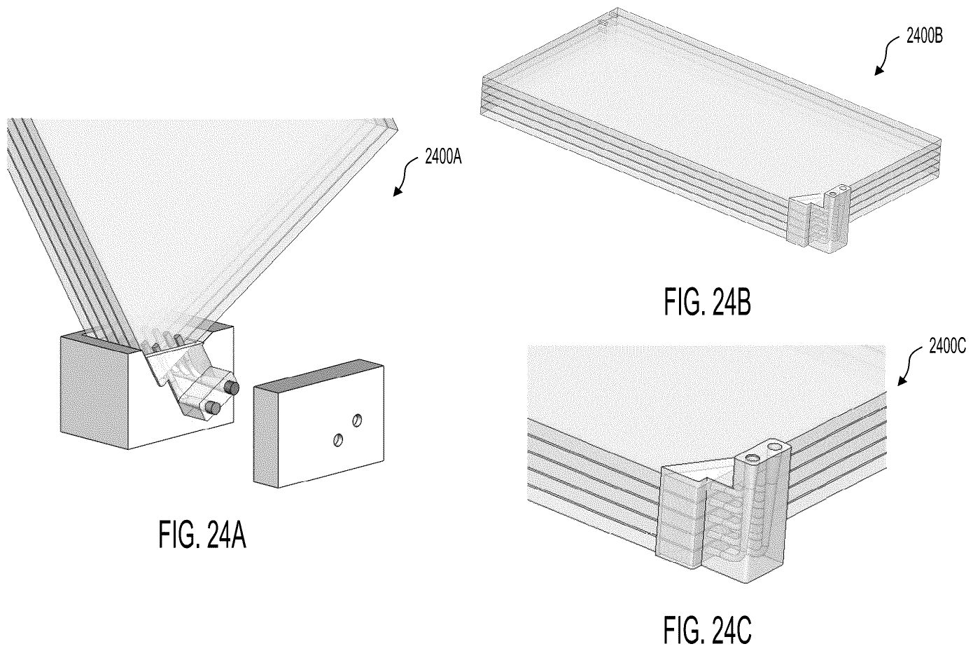

FIGS. 24A, 24B, and 24C show the final steps in the example process for manufacturing the alternative edge manifold, according to an embodiment. A suitable material, such as PDMS, can then be poured into the mold and cured. The mold can then be removed, as shown in the view 2400A, and the negative space manifolds can be melted, dissolved, chemically broken down, or otherwise removed from the inside of the cast manifold, as shown in the views 2400B and 2400C of FIGS. 24B and 24C. Because the interior constructs were manufactured to be smooth at a high-resolution, the sidewalls of the cast alternative edge manifold are also smooth, and result a desired amount of shear stress on the blood, as described herein.

FIGS. 25A and 25B show views 2500A and 2500B of example ducts that may be utilized as an edge manifold for a device similar to that shown in FIG. 1A, according to an embodiment. As shown, molded plastic or metal rectangular ducts can allow for direct connections to each layer to be made in a manner similar to the circular tubing but with a shorter height, allowing for layers to much thinner and minimizing/eliminating the long entrance region where the flow path slowly transitions from circular to a high-aspect ratio rectangular duct.

In addition to the functionality described herein, the oxygenator device (e.g., an ECMO device similar to the system 100 described in connection with FIGS. 1A-1D, the dual membrane device, etc.) may be utilized as a bioreactor. Bioreactor technologies can utilize precise manipulation of suspended cells such as lymphocytes in flow streams, focusing on precision control over flow, shear and velocity, management of cell concentrations in the fluid, controlled addition of oxygen and nutrients and removal of waste products, and scaling of the technology in a robust and miniaturized form factor. The devices described herein can include these attributes, and therefore the devices described herein can be utilized as a bioreactor for applications in cell therapy and regenerative medicine.

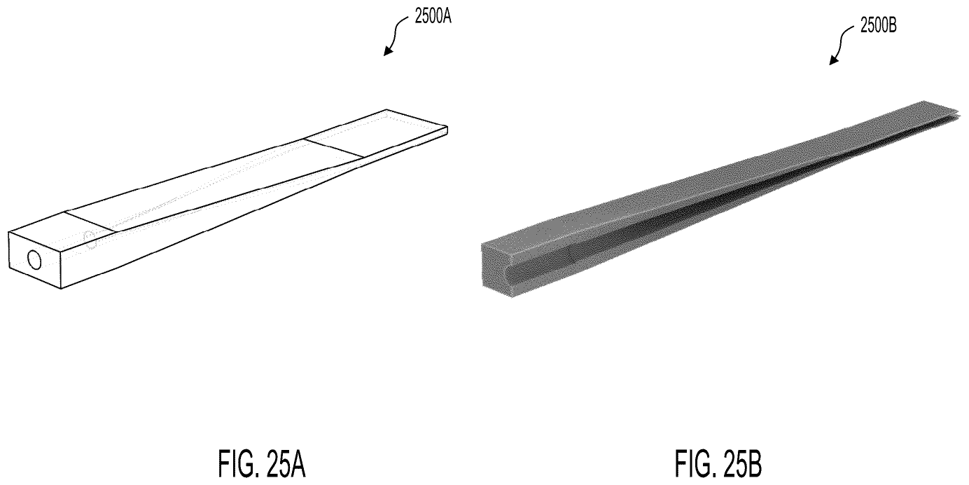

FIGS. 26A-29C provide example experimental results from various experiments performed using systems implemented based on the techniques described herein. The results of these experiments should not be considered limiting to the claims. FIGS. 26A and 26B show graphs of example experimental results from tests measuring oxygen transfer at different blood flow rates, according to an embodiment. Single layer device performance was evaluated by quantifying the transfer of oxygen into the blood channel over a range of blood flow rates. Blood oxygenation levels were measured at discreet flow rates ranging from 25 mL/min to 150 mL/min and compared to predicted results from computational modeling. The oxygen transfer rate (mL O₂/min) into the blood channels increased as blood flow rate increased, reaching a peak average of ˜3 mL O₂/min, as shown in FIG. 26A at 100 mL/min blood flow. Additional oxygen pressure applied to the oxygen channel increased the O₂ transfer rate to above 4 mL O₂ mL/min at 100 mL/min blood flow rate, and maintained the transfer rate as blood flow increased to 150 mL/min. FIG. 26B illustrates the volume percent (Vol %, volume of O₂ transferred into a unit volume of blood) of oxygen in the blood at each flow rate. Vol % was highest at low flow rates as the blood resident time is highest. At 100 mL/min of blood flow through the device, an average 3.16 Vol % oxygen transfer was measured with no applied backpressure and 4.7 Vol % was measured with applied backpressure. Vol % transfer remained above 3.2 Vol % at blood flow rates of 150 mL/min with applied backpressure in the oxygen channel.

As shown in FIGS. 26A and 26B, transfer of oxygen into the blood channel of the device was measured over a range of flow rates and compared to predicted values. FIG. 26A shows that the oxygen transfer rate increased with increased blood flow and with increased applied backpressure, reaching over 5 mL O2/min at the maximum conditions tested. FIG. 26B shows that he volume of oxygen transferred into a unit volume of blood (Vol %) remains above 4 Vol % at a nominal flow rate of 100 mL/min. Vol % remains above 4.2 Vol % when additional oxygen pressure is applied in the oxygen channel. N=2-6 replicates for each data point. Dotted lines represent predicted transfer of oxygen based on simulation results.

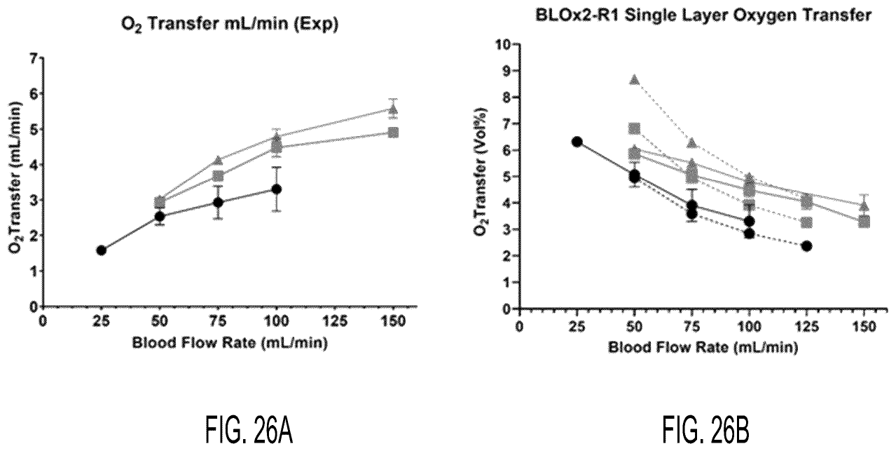

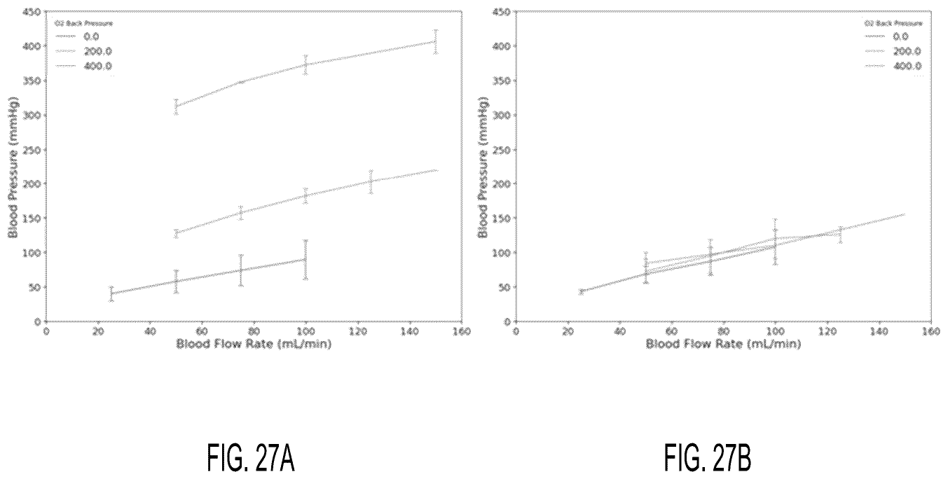

FIGS. 27A and 27B show graphs of example experimental results from tests measuring blood pressure at different blood flow rates, according to an embodiment. Pressure in the blood layer was monitored during oxygen transfer tests at each blood flow rate and applied oxygen backpressure. The multi-banked design and channel dimensions of the example system resulted in a pressure drop under 60 mmHg at 100 mL/min with no applied backpressure. The addition of applied backpressure significantly increased the pressure drop measured in the blood channel by over 10×, likely due to membrane bowing in the on-layer blood distribution manifolds. To address this, a design modification can be incorporated into an ECMO device, as described in connection with FIGS. 8A-8C, to mitigate high pressures recorded in FIG. 27A. Data in FIG. 27B illustrates the significant reduction of pressure measured in the blood channel at applied backpressures, where the maximum can pressure drop at 0, 200 and 400 mmHg applied backpressures was 105 mmHg, 120 mmHg and 135 mmHg respectively at 100 mL/min blood flow.

As shown in FIGS. 27A and 27B, pressure recorded in the blood layer depends on flow rate, applied oxygen backpressure, and oxygen layer design. FIG. 27A shows that the pressure drop remains below 100 mmHg when there is no additional backpressure applied to the oxygen channel. In the nominal design of the oxygenator, the blood pressure drop increases with added backpressure. FIG. 27B shows that modifications to the oxygen layer design eliminate membrane bow in fingers and trunk regions of the device, collapsing pressure drop in the blood channel across applied backpressures.

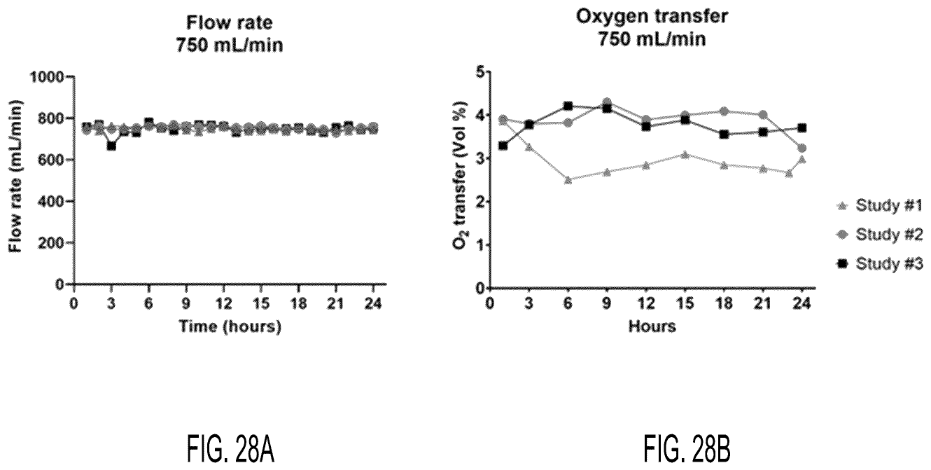

FIGS. 28A and 28B show graphs of example experimental results from tests measuring flow rate and oxygen transfer, respectively, over time in animal studies, according to an embodiment. To gather these example results, three 8-layer microfluidic oxygenator devices were evaluated for 24 hours in a porcine animal study with all three lasting the duration of the study. Target blood flow rate was 750 mL/min and predicted oxygen transfer was 3 Vol % with no applied oxygen backpressure. As shown in FIGS. 28A and 28B, all three studies maintained the target flow rate for 24 hours and transmembrane oxygen transfer was relatively stable throughout the duration of the study, ranging between an average of 3 and 4 Vol % and beating predicted values.

FIGS. 28A and 28B show that all three example 8-layer microfluidic oxygenators survived 24 hour animal studies. FIG. 28A shows that the flow rate maintained a target of 750 mL/min for 24 hours. FIG. 28B shows that the oxygen transfer in study #1 was around 3 vol % compared to around 4 vol % in studies #2 and #3, each relatively stable for 24 hours, indicating stable performance of the device over time.

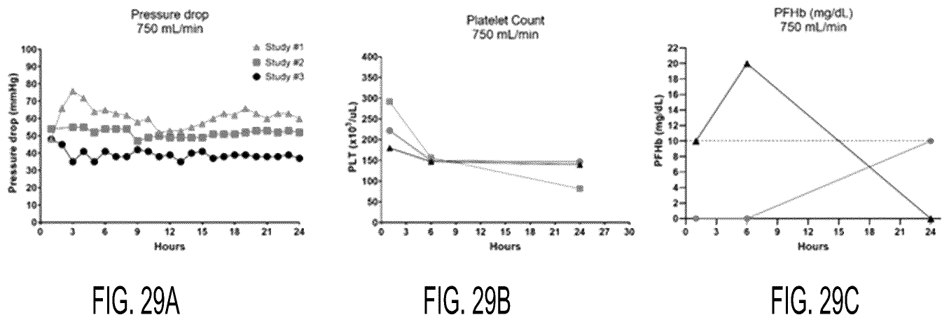

FIGS. 29A, 29B, and 29C show graphs of example experimental results indicating blood health from animal tests, according to an embodiment. Additional readouts during the in vivo animal studies included pressure drop in the blood channel, platelet counts measured and plasma-free hemoglobin (PFHb) measured from blood sampled immediately post-device. The pressure drop remained stable for 24 hours for all three studies, as seen in FIG. 29A, although variable across individual circuits. Animal study #1 had a brief spike of blood pressure drop seen at the 3-hour mark, before steadying out around 60 mmHg for the remainder of the study. Studies #2 and #3 were relatively stable around 50 mmHg and 40 mmHg, respectively for 24 hours. As shown in FIG. 29B, platelet count tended to decrease within 6 hours of the study initiation with a characteristic plateau by 24 hours. As shown in FIG. 29C, PFHb was generally below the limit of detection for all three studies except one measurement in Study #3 of 20 mg/dL at the 6-hour mark. The subsequent measurement in the same study was 0 mg/dL at the 24-hour time point. The PFHb measured in studies #1 and #2 were identical over time, with all three time points at or below limit of detection, indicating little to no injury to red blood cells as collected immediately post-device.

FIGS. 29A, 29B, and 29C show readouts indicative of blood health were measured over 24 hours for each of the three animal studies. FIG. 29A shows that pressure drop was measured in the vascular channel. FIG. 29B shows platelet count and FIG. 29C shows plasma-free hemoglobin, which were measured in blood sampled immediately post-device. The dotted line in FIG. 29C represents the limit of detection for PFHb.

Further experiments were conducted using the ECMO systems described herein to compare the efficacy and safety of the ECMO of the present disclosure with those of traditional HFMO systems. The in vivo experiment involved an experimental group of three pigs being treated using the ECMO devices described herein and a control group of three pigs being treated using traditional HFMO devices. The results of said experiments show that the systems described herein have superior efficacy and safety compared to traditional HFMO systems. The three 3D bioinspired microfluidic oxygenators (e.g., the ECMO devices described herein) at 1.2 L/min blood flow rate, when compared the three HFMO devices in the control group showed a distinct advantage for the ECMO devices based on the key performance parameter PFR (ratio of PaO₂, arterial oxygen tension, to FiO₂, the fraction of oxygen in the inspired ventilator gas stream).

In the example experiment, the first animal for each group succumbed early, due to the injury combined with high anticoagulation protocols. The next two animals in each group experienced reduced heparin levels, while the microfluidic ECMO devices described herein maintained PFR in the mild acute respiratory distress syndrome (ARDS) (PFR>200) or healthy (PFR>300) range. Of the two animals in the control groups treated using traditional HFMO devices, one animal died early (24 h) and for the other, the PFR remained in the severe ARDS range. Further analysis of hemocompatibility data shows a distinct advantage with respect to resistance across the three devices that survived till end of test. During the experiments, the resistance in the ECMO devices remained stable while the resistance in the corresponding HFMO devices experienced a rapid increase over time. Moreover, the ECMO devices described herein do not necessarily utilize an antithrombotic coating, which improves overall hemocompatibility and is more amenable to reduced systemic anticoagulation protocols.

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements.

The devices, systems, and methods described herein may be embodied in other specific forms without departing from the characteristics thereof. The foregoing implementations are illustrative rather than limiting of the described devices, systems, and methods. Scope of the devices, systems, and methods described herein is thus indicated by the appended claims, rather than the foregoing description, and changes that come within the meaning and range of equivalency of the claims are embraced therein.

What is claimed is:

1. An extracorporeal membrane oxygenation (ECMO) device, comprising:
a first layer defining a plurality of banks of first channels each extending in a first direction, the plurality of banks of first channels configured to receive blood via a trunk channel;
a second layer defining a bank of second channels extending in a second direction, the bank of second channels configured to receive oxygen, the first direction different from the second direction; and
a membrane disposed between the first layer and the second layer and configured to cause the oxygen to permeate from the second layer to the first layer to oxygenate the blood.

2. The ECMO device of claim 1, wherein at least one bank of the plurality of banks of first channels is configured to receive the blood via a finger channel in fluid communication with the trunk channel, the finger channel having a taper along a length of the at least one bank.

3. The ECMO device of claim 2, wherein the at least one bank comprises a plurality of bifurcating channels in fluid communication with the finger channel.

4. The ECMO device of claim 3, wherein each of the plurality of bifurcating channels bifurcate at least twice.

5. The ECMO device of claim 1, wherein the trunk channel comprises at least one trunk ramp configured to maintain shear stress on the blood within a predetermined range as the blood flows through the plurality of banks of first channels.

6. The ECMO device of claim 1, wherein the bank of second channels is configured to receive the oxygen via an oxygen channel having a taper along a length of the bank of second channels.

7. The ECMO device of claim 6, wherein the oxygen channel comprises one or more support structures.

8. The ECMO device of claim 1, wherein the membrane has a thickness ranging from about 50 μm to about 100 μm.

9. The ECMO device of claim 1, wherein the membrane comprises polydimethylsiloxane (PDMS).

10. The ECMO device of claim 1, wherein the plurality of banks of first channels comprise four banks of first channels, each receiving oxygen from the bank of second channels via the membrane.

11. A system, comprising:
a housing comprising a plurality of oxygenator layers, each of the plurality of oxygenator layers comprising:
a bank of first channels configured to receive blood,
a bank of second channels configured to receive oxygen, and
a membrane configured to cause the oxygen to permeate from the bank of second channels to the bank of first channels to oxygenate the blood; and
a vertical manifold configured to provide the blood to each of the plurality of oxygenator layers.

12. The system of claim 11, wherein the vertical manifold is configured to maintain a range of shear stress in the blood ranging from about 7 dynes/cm² to about 35 dynes/cm².

13. The system of claim 11, wherein the vertical manifold is further configured to provide the oxygen to the plurality of oxygenator layers.

14. The system of claim 11, wherein the housing comprises a plurality of trays each having a respective oxygenator layer of the plurality of oxygenator layers, wherein the vertical manifold is coupled to the plurality of trays.

15. The system of claim 11, further comprising an oxygen source configured to provide the oxygen at a predetermined pressure.

16. The system of claim 11, wherein the vertical manifold comprises a plurality of tubes positioned within a casing.

17. A layer of an extracorporeal membrane oxygenation (ECMO) device, comprising:
an inlet configured to receive blood;
a trunk channel in fluid communication with the inlet;
a plurality of finger channels extending from the trunk channel; and
a plurality of banks of blood channels, each of the plurality of banks of blood channels in fluid communication with a respective one of the plurality of finger channels via a bifurcating manifold configured to maintain shear stress on the blood within a predetermined range.

18. The layer of claim 17, wherein a shape of one or more curves of the plurality of finger channels is configured to maintain the shear stress on the blood within the predetermined range.

19. The layer of claim 17, wherein each of the plurality of banks of blood channels are in fluid communication with an outlet channel via a second bifurcating manifold.

20. The layer of claim 19, wherein the outlet channel is parallel to the trunk channel.
